(19) United States
(12) Patent Application Publication
(10) Pub. No.: US 2022/0259258 A1
(43) Pub. Date: Aug. 18, 2022

(54) A Manufacturing Process for Peptide and Protein Production

(71) Applicant: MyTide Therapeutics, Inc., Boston, MA (US)

(72) Inventors: Justin Alexander MacDonald Lummiss, Cambridge, MA (US); Chase R. Olle, Allston, MA (US); Kevin Shi, Boston, MA (US); Dale Arlington Thomas, III, Hampden, ME (US)

(21) Appl. No.: 17/618,781

(22) PCT Filed: Jun. 12, 2020
(86) PCT No.: PCT/US2020/037441

§ 371 Date: Dec. 13, 2021

Related U.S. Application Data
(60) Provisional application No. 62/861,821, filed on Jun. 14, 2019.
(60) Provisional application No. 63/009,563, filed on Apr. 14, 2020.

(51) Int. Cl. C07K 1/04

(52) U.S. Cl. CPC C07K 1/042; C07K 1/045

(57) ABSTRACT

The present disclosure provides coupling methods and systems for producing peptides or proteins using semi-continuous or continuous manufacturing techniques to enable rapid production of peptides and proteins using solid phase peptide synthesis (SPPS). The disclosure provides a higher degree of process control for peptide and protein product manufacturing using semi-continuous or continuous manufacturing techniques with inline analytics and automation.

### RELATED APPLICATION(S)

This application claims the benefit of U.S. Provisional Application No. 62/861,821, filed on Jun. 14, 2019 and claims the benefit of U.S. Provisional Application No. 63/009,563 filed on Apr. 14, 2020.

The entire teachings of the above application(s) are incorporated herein by reference.

### GOVERNMENT SUPPORT

This invention was made with government support under Grant No. 1938756 awarded by the National Science Foundation. The government has certain rights in the invention.

### TECHNICAL FIELD

The present disclosure relates to semi-continuous or continuous manufacturing techniques to enable rapid production of peptides and proteins using solid phase peptide synthesis (SPPS). In particular, the coupling method and system uses semi-continuous or continuous manufacturing techniques with inline analytics which facilitates a higher degree of process control for peptide and protein product manufacturing.

### BACKGROUND

Peptides and proteins play a vital role by mediating an extensive range of biological processes, acting as signaling molecules, antibiotics or hormones. Due to the highly specific interaction with their biological targets, peptides and proteins have been widely used in medicine and represent a growing subset of the therapeutic market. To supply the need for therapeutic peptides and proteins, standard batch chemistry or semi-batch methodologies utilizing solid phase peptide synthesis (SPPS) has allowed for advancements in many areas of peptide and protein research, most notably for customized and personalized treatments. However, standard batch chemistry or semi-batch methodologies, which are accomplished through multistep reactions, require the removal of one reactant followed by the addition of another reagent causing longer processing time, reduced yields and therefore lead to decreased process efficiency. Moreover, batch reactions are difficult and time-consuming to optimize due to the lack of inline process analytical techniques that can provide real-time manipulation of continuous and discrete variables, e.g., the number of times the synthetic step is repeated, within the system. Therefore, an improved manufacturing process for solid phase peptide synthesis (SPPS) that can provide real-time manipulation of semi-continuous or continuous variables is needed.

### SUMMARY

Provided herein are methods and systems for producing a peptide or protein using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin, and thus facilitates a higher degree of process control for peptide and protein product manufacturing. These methods and systems enable controlled and efficient synthesis of longer length complex peptides due to the ability to limit mixing and diffusion time allowing the potential to dramatically decrease waste during solid phase peptide synthesis (SPPS). Moreover, these semi-continuous or continuous manufacturing techniques along with inline analytics and real-time feedback facilitate higher degrees of control for efficient peptide and protein manufacturing. As used herein, “real-time” feedback relating to one or more process variables is used to control the method or system. In some embodiments, the methods or systems are automated and is used in a high throughput system, such as a continuous flowing stream of material to be treated, or optionally segmented, e.g., semi-continuous. In other embodiments, the method or system is performed in one or more tubes or any other continuous system as described herein.

In one aspect, the disclosure provides a method for producing a peptide using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin.

In another aspect, the disclosure provides a method for producing a protein using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid, peptide or protein that is immobilized on the resin.

The present disclosure also provides herein a system for producing a peptide or protein using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin.

In one aspect, the disclosure provides a system for producing a peptide using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin.

In another aspect, the disclosure provides a system for producing a protein using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid, peptide or protein that is immobilized on the resin.

The present methods and systems may be useful for producing custom peptides or proteins. In preferred embodiments, the methods use semi-continuous or continuous manufacturing comprising inline analytics and solid phase peptide synthesis (SPPS) enabling controlled production of peptides or proteins. In some embodiments, software control is employed to enable automation of the process. In other embodiments, inline analytics are incorporated to optimize the process for a step-by-step amino acid/peptide-amino acid/peptide synthesis. In certain embodiments, the methods and systems employ automated technology (e.g., robotics) and inline analytics (e.g., hardware/software assemblies, modules, and the like) to optimize and scale the process for the production peptides or proteins.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows a scheme for truncation of a growing peptide chain caused by unreacted activator reaching the synthesis vessel, e.g., the resin.

FIG. 1B shows a scheme for a productive reaction involving an activated amino acid.

FIG. 2 shows a scheme for deactivation of an activating agent when the complex is not reacted with an amino acid prior to delivery to the resin.

FIG. 3 shows an illustration of different slugs of material for performing peptide couplings.

FIG. 4 shows an illustration of a solid phase slug flow (SPSF) synthesis platform according to some embodiments of the disclosure.

FIG. 5 shows an illustration of a solid phase slug flow (SPSF) synthesis platform according to certain embodiments of the disclosure.

FIG. 6 shows a scheme for an asynchronous time line of slug reagent preparation and delivery to the peptide resin bed.

FIG. 7 shows an image of a robotic flow chemistry system for completing peptide synthesis installed on a 12″×18″×18″ optical breadboard.

FIG. 8 shows a graph of the inline process analytical data obtained from the reactor outlet.

FIG. 9 shows a chromatogram of acyl carrier protein (ACP(65-74)) at >92% purity.

FIG. 10 shows a chromatogram of the inline UV data from the reactor outlet.

FIG. 11 shows a chromatogram of slug formation.

FIG. 12 shows a chromatogram of no slug formation and slug formation in one embodiment.

FIG. 13 shows a chromatogram of no slug formation and slug formation in another embodiment.

FIG. 14 shows a chromatogram of acyl carrier protein (ACP(65-74)).

FIG. 15 shows a chromatogram of pexiganan.

FIG. 16 shows a chromatogram of glucagon-like-peptide-1 (GLP-1).

FIG. 17 shows an image of pexiganan bioequivalence.

FIG. 18 shows a chromatogram of a 102-mer protein.

### DETAILED DESCRIPTION

A description of example embodiments follows.

Solid phase peptide synthesis methods and associated systems using semi-continuous or continuous manufacturing to deliver a plurality of reagents to a resin are described. Solid-phase peptide synthesis (SPPS) has become the predominant manufacturing methodology for small and large quantities of peptides. Solid-phase peptide synthesis is a process in which amino acids or peptides are added to amino acids, peptides or proteins that are immobilized on a solid support, e.g., resin. This technique utilizes standard batch chemistry or semi-batch methodologies to grow a peptide chain on a resin support. Growth of the peptide chain is achieved through repeated cycles of coupling where the deprotection step expose an active site of the amino acid, peptide or protein that are immobilized on the resin, and the coupling step forms new amide bonds at the exposed active sites. Yield, purity, and efficiency in SPPS are highly dependent on the ability to consistently deliver the required plurality of reagents to each particular active site on the amino acids, peptides or proteins that are immobilized on the resin support, and then completely remove excess reagents and reaction by-products from the resin support before introducing the plurality of reagents for subsequent steps. In standard batch chemistry or semi-batch methodologies, which are accomplished through multistep reactions, results in long processing times, reduced yields and decreased process efficiencies. Moreover, batch production is often difficult and time-consuming to optimize due to limitations in mass and heat transport, which lead to non-homogeneous rates of reactions within batch reactors that limit the ability to provide real-time manipulation of continuous variables to optimize the system. In addition, heterogeneous batch reactions present an additional challenge for inline process analytical technologies which are capable of providing real-time insight into the bulk characteristics of the solid material. Thus, the use of inline analytics with real-time feedback allows the ability to scale flow reactions for the manufacturing of small and large quantities of peptides or proteins.

The present disclosure generally relates to methods and systems for producing a peptide or protein using semi-continuous or continuous manufacturing comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin. In such embodiments, the disclosure enables controlled delivery of a plurality of reagents for the production of peptides or proteins during liquid or solid phase peptide synthesis. In some embodiments, the methods and systems can be used to perform solid phase peptide synthesis quickly while maintaining high yields and reaction efficiency. Certain embodiments relate to methods and systems that may be used to heat, transport, and/or mix a plurality of reagents in ways that reduce the amount of washout, mixing, and/or coupling time, e.g., reaction time, required to perform solid phase peptide synthesis for producing peptides and proteins. In other embodiments, the methods and systems can be used to provide efficient mixing of activator and amino acid or peptide, and to ensure complete reaction conversion prior to the plurality of reagents passing through the resin. The methods and systems as described herein, are easily scalable, can be carried out efficiently, at low cost.

In addition to using manually controlled transfer techniques of chemical reagents, the methods and systems for producing a peptide or protein using semi-continuous or continuous manufacturing incorporates automatically or robotically controlled techniques to transfer chemical reagents. Advantageously the methods and systems of the present disclosure can be carried out using automated and robotic liquid transfer systems. In some embodiments, the methods and systems for producing a peptide or protein using semi-continuous or continuous manufacturing can be performed using robotics, e.g., high-throughput robotics. High-throughput robotics are particularly useful when transferring chemical reagents or agents from chemical compound libraries and the such. In other embodiments, the methods and systems for producing a peptide or protein using semi-continuous or continuous manufacturing can be performed using automation.

Current slug flow techniques for automated synthesis generally cannot measure real-time reaction progress and rates for each step of the synthesis process which results in inefficiencies in automation causing unoptimized mixing and reaction times, excess stoichiometries, and excess wash volumes. Moreover, the current systems in the art are generally limited to a two reagent process and heterogeneous chemistry, which hinder the mixing of reagents due to the physical limitations which have prevented its widespread use for solid phase peptide synthesis (SPPS).

In certain embodiments, the semi-continuous or continuous manufacturing as disclosed herein, comprises slug flow. A slug is formed by encapsulating reagents between two immiscible fluids or gases. In certain preferred embodiments, the methods and systems for producing a peptide or protein uses slug flow comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid or peptide that is immobilized on the resin. These methods and systems described herein, enable controlled and efficient synthesis of longer length complex peptides due to the ability to limit mixing and diffusion time to a slug, allowing the potential to dramatically decrease waste during solid phase peptide synthesis (SPPS). In particular, a plurality of reagents is mixed as slugs and delivered to the reactor, e.g., resin, as slugs. Heating can be applied to a slug to increase the reaction kinetics. In other embodiments, the methods and systems can be used to provide efficient mixing of activator and amino acid or peptide in a slug, which ensures complete reaction conversion prior to the plurality of reagents passing through the resin.

Due to low dispersion and length scales which are generally related to slug flow technologies, inline analytical technologies are described herein, which allow real-time optimization and decision making during solid phase peptide synthesis (SPPS). The present disclosure also generally relates to slug flow systems that provide cycle-to-cycle (amino-acid-to-amino-acid) and step-by-step feedback and control which allows the use of minimally forcing conditions for each synthetic step. In certain embodiments, the use of minimal forcing conditions provides the ability to increase purity by eliminating unnecessary exposure of the growing peptide chain to harsh reaction conditions. In such embodiments, the system provides the ability to optimize peptide and protein synthesis for maximum efficiency by decreasing solvent and reagent volume which results in increased crude product purity, thus, reducing the need for downstream purification.

The semi-continuous or continuous manufacturing processes according to the present disclosure is advantageous in that it has one or more of the following benefits: increases the efficiency of manufacturing peptides and proteins by reducing time spent in manufacturing campaigns, which in turn reduces cost of deliverables, and also reduces the complexity of the manufacturing process.

As described herein, slug flow for solid phase peptide synthesis, e.g., solid phase slug flow (SPSF), enables easy integration for automation and robotics which allows for precision reaction kinetics, excellent mixing control, efficient coupling transformations, and inline analytics for real-time monitoring of reactions and process steps. In some embodiments, the method and system allows the incorporation of immediate feedback, e.g., before the next reaction reagents that enter into the flow stream headed to the reactor, e g., resin, and responses into the automated and robotic system enabling algorithms to intelligently manipulate different continuous variables (e.g., double coupling, double deprotection, temperature, time, and concentration) based on data models for optimal synthesis.

In some embodiments of the disclosure, the methods and systems described herein, introduce a separation media at critical points in the synthesis process, which isolate key reagents into “slugs” between pockets of separation media, e.g., immiscible fluids air/water, oil/water or the like, which eliminates unwanted dispersion and mixing of reagents thereby creating the series of tightly controlled microreactors that enable real-time process optimization of the synthesis process.

In other embodiments, the methods and systems described herein, can decrease waste enabling decreased cost for high value molecular additions. In some embodiments, the methods and systems can produce pure product, thereby reducing the need for post synthesis work-up or purification, e.g., chromatography, which can be highly solvent and time intensive. In certain embodiments, the methods and systems can decrease dispersion resulting in higher control over reducing the amount of solvent, e.g., DMF, required for washing between each step in the coupling process. In still other embodiments, the methods and systems when using precious reagents, e.g., non-canonical amino acids or amino acids with a fluorescent tag, can decrease dispersion resulting in higher control of stoichiometry, thereby reducing the need for large excess of expensive reagents. In certain other embodiments, the methods and systems described herein, decreases the overall synthesis time to produce a peptide or protein.

In some embodiments, the methods and systems described herein, uses automated flow technology and inline analytics to optimize the coupling of amino acids or peptides to the growing amino acid, peptide or protein chain. In other embodiments, the methods and systems use inline analytics which result in real-time feedback which enables less dispersion of the plurality of reagents resulting in higher concentrations of reagents prior to contacting the unprotected amino acid, peptide or protein that is immobilized on the resin. In certain embodiments, the signal time for each particular reagent observed by a plurality of detectors can be shorter. In preferred embodiments, the methods and systems comprising inline analytics has the ability to provide a stronger signal in less time to create “real-time” control over the entire synthetic process.

In other embodiments, the methods and systems described herein, implements the use of inline analytics to provide real-time data to assess a successful deprotection process and to a subsequent coupling step, allowing a decrease in process time. “Real-time” feedback is the ability to make a decision before the next reaction step is initiated, e.g., when the reagents are added to the flow stream headed to the resin. Based on “real-time” feedback for a deprotection process, the user can decide whether to repeat the deprotection process to insure complete deprotection or to proceed under modified deprotection parameters, e.g., using forcing conditions, to insure complete deprotection.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the methods and systems disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₋₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In other embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucin (Leu), isoleucin (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro. As described herein, a “side chain of an amino acid” includes a protecting group-containing side chain of an amino acid. For example, the protecting group may include, but is not limited to, 9-fluorenylmethyl carbamate (Fmoc), t-butyl carbamate (Boc), benzyl carbamate, acetamide, or trifluoroacetamide. In other embodiments, the protecting group is acetamidomethyl (Acm), acetyl (Ac), adamantyloxy (AdaO), benzoyl (Bz), benzyl (Bzl), 2-bromobenzyl, benzyloxy (BzlO), benzyloxycarbonyl (Z), benzyloxymethyl (Bom), 2-bromobenzyloxycarbonyl (2-Br-Z), tert-butoxy (tBuO), tert-butoxycarbonyl (Boc), tert-butoxymethyl (Bum), tert-butyl (tBu), tert-butylthio (tButhio), 2-chlorobenzyloxycarbonyl (2-Cl-Z), cyclohexyloxy (cHxO), 2,6-dichlorobenzyl (2,6-DiCl-Bzl), 4,4′-dimethoxybenzhydryl (Mbh), 1-(4,4-dimethyl-2,6-dioxo-cyclohexylidene) 3-methyl-butyl (ivDde), 4-{N-[1-(4,4-dimethyl-2,6-dioxo-cyclohexylidene)3-methylbutyl]-amino) benzyloxy (ODmab), 2,4-dinitrophenyl (Dnp), fluorenylmethoxycarbonyl (Fmoc), formyl (For), mesitylene-2-sulfonyl (Mts), 4-methoxybenzyl (MeOBzl), 4-methoxy-2,3,6-trimethyl-benzenesulfonyl (Mtr), 4-methoxytrityl (Mmt), 4-methylbenzyl (MeBzl), 4-methyltrityl (Mtt), 3-nitro-2-pyridinesulfenyl (Npys), 2,2,4,6,7-pentamethyldihydrobenzofurane-5-sulfonyl (Pbf), 2,2,5,7,8-pentamethyl-chromane-6-sulfonyl (Pmc), tosyl (Tos), trifluoroacetyl (Tfa), trimethylacetamidomethyl (Tacm), trityl (Trt) or xanthyl (Xan).

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written herein. References to an element in the singular is not intended to mean “one and only one” unless specifically stated, but rather “one or more”. Unless specifically stated otherwise, terms such as “some” refer to one or more, and singular terms such as “a”, “an” and “the” refer to one or more.

The term “semi-continuous manufacturing” means a process which continuously feeds a fluid comprising a plurality of reagents through at least a part of the system, e. g., the flow stream is stopped for a period of time to introduce new reagents or facilitate the completion of a reaction without the reagents flowing through the system. The term “continuous manufacturing” means a process which continuously feeds a fluid comprising a plurality of reagents through the entire system. For example, in any of the exemplary semi-continuous or continuous manufacturing method or system described herein, a liquid medium comprising a plurality of reagents is continuously fed into at least part of the system or the entire system, while it is in operation and results in the coupling of the protected amino acid or peptide to an unprotected amino acid, peptide or protein that is immobilized on the resin. Another example of a semi-continuous or continuous manufacturing method or system described herein, a liquid medium comprising a plurality of reagents is continuously fed into at least part of the system or the entire system, while it is in operation and results in the deprotection of the protected amino acid, peptide or protein that is immobilized on the resin, e.g., the flow stream is stopped to add the slugs in the line of a closed system that runs in a continuous path from the point of mixing across the solid supported bed, e.g., resin. Additional examples include a liquid medium comprising a plurality of reagents is continuously fed into at least part of the system or the entire system, while it is in operation and results in the cleavage of a protected or unprotected amino acid, peptide or protein from the resin.

As used herein, the term “elongation” refers to any decoiling of the secondary or tertiary structure. Decoiling of the secondary or tertiary structure provides easier access to otherwise sterically hindered reactive sites on the growing peptides.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosins, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from between about 5 to about 100 amino acids in length. In some embodiments, the peptide is from between about 40 to about 100 amino acids in length.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain about 101 to about 1000 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g., keratin), as enzymes, as hormones, as transporters (e.g., hemoglobin), as antibodies, or as regulators of gene expression. Proteins or peptides are required for the structure, function, and regulation of cells, tissues, and organs.

The term “protecting group” as used herein may be understood in the broadest sense as a group which is introduced into a molecule by chemical modification of a functional group to block said group from reaction in subsequent process steps, e.g., to prevent side reactions of the amino acid side chains. Examples of amino protecting groups are the tert-butyloxycarbonyl (Boc) and 9-fluorenylmethyloxycarbonyl (Fmoc) groups. Examples of carboxylic acid protecting groups are unreactive esters such as methyl esters, benzyl esters, or tert-butyl esters.

The term “synthesis” as used herein indicates the forming, growing or building of a more complex peptide or protein from amino acids or simpler peptides. In particular, synthesis in the sense of the present disclosure relates to peptide or protein synthesis indicating a process of reacting amino acids or simpler peptides together in a chemical reaction coupling the amino acids or simpler peptides to form a more complex peptide or protein. Solid phase peptide synthesis (SPPS) incorporates several steps that are repeated as additional amino acids are added to a growing peptide chain. The term “solid phase” refers to resin particles to which initial amino acids, and then the growing peptide chains, are attached. Because the chains are attached to resin particles, the chains can be handled as if they were a collections of solid particles, particularly for the washing steps, separation steps, and the filtration steps, thus, making the overall synthesis process easier, in many cases, than pure solution synthesis. SPPS as described herein, can be carried out on gel phase or solid phase supports. Suitable resins and linkers may be based on polystyrene, polystyrene-PEG composites, PEG, PEGA, cross-linked ethoxylate acrylate (CLEAR), polyamides, polydimethylacrylamide, or any other support with the desired physical and chemical properties known to the skilled artisan. Resins and linkers based on beaded polystyrene with 1% divinylbenzene can be used, which typically have a size distribution of 200-400 mesh, 400-600 mesh, 100-200 mesh, 100-400 mesh, or 400-600 mesh. Polystyrene based 4-alkoxybenzyl alcohol (Wang) resin, diphenyldiazomethane (PDDM) resin, 4-(2′,4′-dimethoxyphenyl-Fmoc-aminomethyl)-phenoxymethyl-polystyrene (Rink) resin, 2-methoxy-4-alkoxybenzyl alcohol (Sasrin) resin, 4-hydroxymethyl-3-methoxyphenoxybutyric acid (HMPB) and 2-chlorotrityl chloride (CTC) resin are suitable for use with the methods of the present disclosure and are commercially available from suppliers such as Sigma-Aldrich, Bachem and EMD Millipore. However, any other resin or photo cleavable linkers known to the skilled artisan that is suitable for SPPS may be used herein.

As used herein, the phrases “selected from the group consisting of,” “chosen from,” and the like, include mixtures of the specified materials. Where a numerical limit or range is stated herein, the endpoints are included. Also, all values and subranges within a numerical limit or range are specifically included as if explicitly written out. References to an element in the singular is not intended to mean “one and only one” unless specifically stated, but rather “one or more.” Unless specifically stated otherwise, terms such as “some” refer to one or more, and singular terms such as “a,” “an” and “the” refer to one or more.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

### Methods and Systems of the Disclosure

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted. As used herein, the phrase “and/or” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The basic principle for solid phase peptide synthesis (SPPS) is a stepwise addition of amino acids to a growing peptide chain anchored via a linker molecule to a solid phase support, typically a resin particle, which allows for cleavage and purification once the polypeptide chain is complete. In certain embodiments, a solid phase resin support and a starting amino acid are attached to one another via a linker molecule. Such resin-linker-acid matrices, photo cleavable, or acid/base labile are commercially available. The amino acid to be coupled to the resin is protected at its N-terminus by a chemical protecting group. The amino acid may also have a side-chain protecting group. Such protecting groups prevent undesired or deleterious reactions from taking place during the process of forming the new peptide bond between the carboxyl group of the amino acid to be coupled and the unprotected N-amino group of the peptide chain attached to the resin. The amino acid to be coupled is reacted with the unprotected N-amino group of the N-terminal amino acid of the peptide chain, increasing the chain length of the peptide chain by one or more amino acids. The carboxyl group of the amino acid to be coupled may be activated with a suitable chemical activating agent to promote reaction with the N-amino group of the peptide chain. The N-protecting group of N-terminal amino acid of the peptide chain is then removed in preparation for coupling with the next amino acid residue. This technique consists of many repetitive steps making automation and robotics attractive whenever possible. Those skilled in the art will appreciate that peptides may be coupled to the N-amino group of the solid phase bound amino acid or peptide instead of an individual amino acid, for example where a convergent peptide synthesis is desired. When the desired sequence of amino acids is achieved, the peptide is cleaved from the solid phase support at the linker molecule. It will be appreciated by those skilled in the art that coupling an amino acid or a peptide to another amino acid or peptide as described herein may comprise forming a peptide bond between the N-terminus of the amino acid or an amino acid of the peptide of one coupling partner and the C-terminus of the amino acid or an amino acid of the peptide of the other coupling partner.

In some embodiments as disclosed herein, provides a method or system for producing a peptide or protein using semi-continuous or continuous manufacturing comprising inline analytics and solid phase peptide synthesis (SPPS). In other embodiments, the semi-continuous or continuous manufacturing comprises slug flow. In still other embodiments, the peptide or protein is produced using semi-continuous manufacturing, wherein the semi-continuous manufacturing comprises slug flow. In certain other embodiments, the peptide or protein is produced using continuous manufacturing, wherein the continuous manufacturing comprises slug flow.

As described herein, solid phase slug flow (SPSF) synthesis is the reaction of a reagent slug with a solid support resin. Slug flow techniques have introduced methods and systems for optimization of single homogenous reaction types with multiple reagents for high-throughput synthesis and reaction condition optimization. In some embodiments, slug flow is generally not a continuous stream, but a liquid-liquid or gas-liquid stream. In other embodiments, liquid-liquid slug separation can be between two immiscible fluids or the liquids can be separated by differences in viscosity or density. In certain embodiments, gas-liquid slugs are generally separated by an inert gas, e.g., air, nitrogen, argon, to prevent reaction between the slug and the separation medium. However, in certain other embodiments, a separation gas that is highly reactive, e.g., hydrogen, may be used to complete a reaction with the reagent slug.

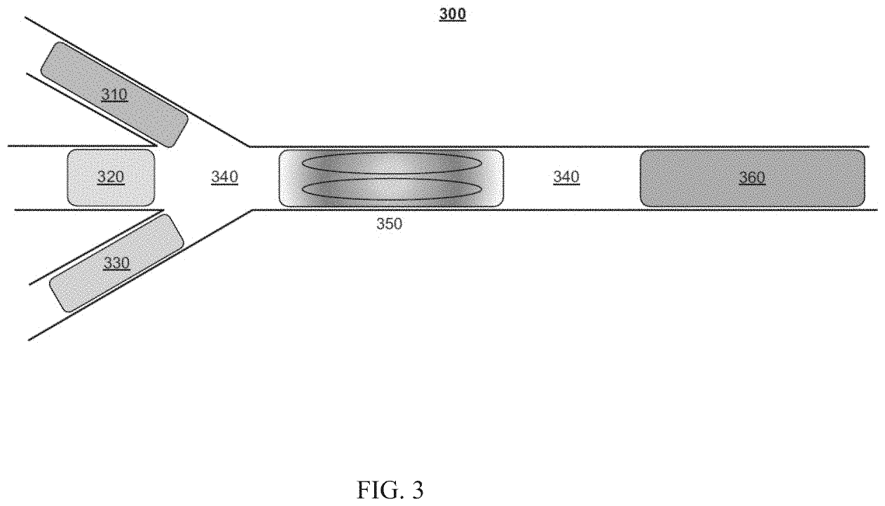

As described herein, “slug flow” or “segmented flow” separates fluids within fluid tubing into discrete sections bounded by the viscous forces of the surrounding material. See FIG. 3. As shown in system 300, the liquid slugs containing amino acid 310, base 320 and activator 330 are flowed through a channel and are separated by separation media 340. The amino acid 310, base 320 and activator 330 are trapped at capture zone 350 where activation of amino acid 310 occurs by mixing. Capture zone 350 is further separated by separation media 340 forming slug 360, which can be carried to an amino acid, peptide or protein that is immobilized on a solid support resin. System 300 enables controlled delivery of reagents for the synthesis of peptides and proteins during solid phase peptide synthesis (SPPS). In some embodiments of the disclosure, solid phase slug flow (SPSF) methods and systems for coupling amino acids or peptides to a growing amino acid, peptide or protein chain overcomes limitations of current batch and continuous flow techniques by isolating the synthesis process into discrete steps. In such embodiments, by discretizing the steps of the process, e.g., dispersion and mixing limitations, the speed, purity, and synthesizable lengths of the peptides or proteins of the methods and systems described herein can be optimized.

In some embodiments, slug flow occurs when the ratio of flow rates for the wetting to the non-wetting phases is close to unity, e.g., at low superficial velocity. At this flow rate the interfacial tension dominates the inertial force and viscous force resulting in the reduction of interfacial area by interfacial tension thereby creating a slug (FIG. 3). In such embodiments, the phase 1 liquid and the phase 2 (liquid or gas) passes the channel alternatively as elongated bubbles with an equivalent diameter larger than that of the channel and appear like slugs. The slug structure is maintained by the interfacial tension, thus, slug coalescence is rarely observed. The Capillary number represents the relative effect of viscous forces versus surface tension acting across an interface between two immiscible liquids or a liquid/gas interface. Based on the Capillary number (Ca), it has been found that three types of slug formation exist: squeezing (10-4<Ca<0.0058), dripping (0.013<Ca<0.1) and transitional (0.0058<Ca<0.013). The dynamics of slug formation is governed by channel geometries, channel properties (e.g., channel type, dimension and hydrophobicity), fluid properties, e.g., density, viscosity, interfacial tension and contact angle, and operating parameters, e.g., pressure, flow rate ratio, temperature and electric field. In other embodiments, the slug forms at low velocity reducing the risk of high pressure flow in the channel device. In such embodiments, the operation of the slug flow technique is useful for enhancing mass transfer. Thus, the slug flow technique described herein, exhibits greater mass transfer performance due to their high degree of stability and their capability to achieve a surface area above 10000 m²/m³ allowing great reproducibility and slug size control.

As described herein, the present disclosure utilizes semi-continuous or continuous manufacturing methods and systems comprising inline analytics and solid phase peptide synthesis (SPPS) to deliver a plurality of reagents to the resin, thereby coupling the protected amino acid or peptide to an unprotected amino acid, peptide or protein that is immobilized on the resin. In certain embodiments, the disclosure utilizes slug flow methods and systems comprising inline analytics and solid phase peptide synthesis (SPPS) to deliver a plurality of reagents to the resin. In certain other embodiments, peptides and proteins are produced using slug flow comprising inline analytics and solid phase peptide synthesis (SPPS), wherein a plurality of reagents is delivered to a resin by semi-continuous or continuous manufacturing, wherein the reagents comprise: a) a protected amino acid or peptide; b) an activating agent; and c) a solvent, thereby coupling the protected amino acid or peptide to an unprotected amino acid, peptide or protein that is immobilized on the resin. In some embodiments, the slug is formed through the merger of three reagent streams, e.g., amino acid, base and activator, into a slug (FIG. 3). In other embodiments, the slugs are separated using a separation medium or reagent, e.g., hydrogen or CO₂. In still other embodiments, the slug flow coupling combines the protected amino acid or peptide, activating agents, and a base into a droplet which flows in a channel to form a slug. In preferred embodiments, the slug flow method and system minimizes dispersion within the flow stream thereby increasing the concentration of the amino acid delivered allowing increased mass transfer.

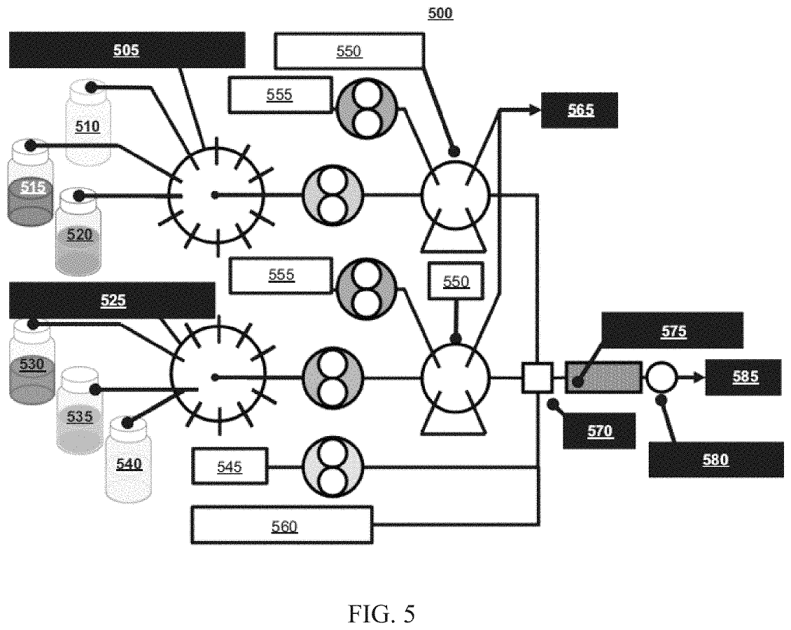

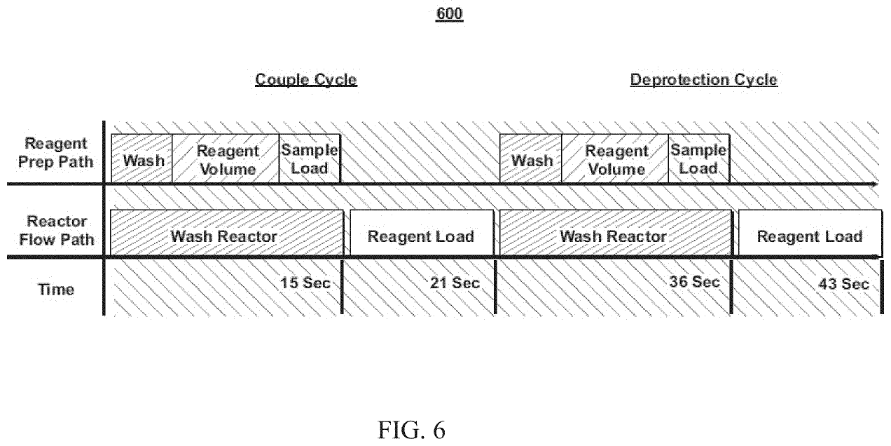

In some embodiments, the present disclosure provides a system 500 that controls mixing time, washout time, separation and reaction time based on slug flow techniques that enable rapid slug formation with real-time process analytics that allow a robust, unattended, high throughput operation for peptide and protein synthesis. See FIG. 5. In certain embodiments, the slugs will be formed in mixer 570 through the combination of reagents (amino acids 510, 515, 520, activators DIC 530, PIP 535, HBTU 540, and base 545) prepared in sample loops 550 that deliver wash solvents 555. In such embodiments, the choice of reagents is selected based on the amino acid selector 505 and the activator selector 525. To isolate the slugs from each other, separation media 560 can be added at the beginning and end of the reagent load step (FIG. 6), encapsulating the slugs within the reagent delivery system. In other embodiments, the separation media 560 can be an inert gas or oil, to limit dispersion and slug mixing within the system. In such embodiments, a slug's internal mixing occurs based on the material interface. In other embodiments, the viscous drag between the moving slug and tube wall causes relative motion of the fluid which is recirculated by the interface resulting in robust internal circulation. In such embodiments, the robust mixing results in a homogenous slug that can be delivered to a reactor containing solid support resin 575 preventing concentration gradients from forming. In still other embodiments, the implementation of conductivity meter 580 into the system can enable the slug flow technology to reduce the amount of waste 565 and 585 generated by the method.

In certain embodiments, the plurality of reagents is delivered to the resin from about 1 to about 60 seconds, e.g., from about 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 57, 58, or 59 seconds. In still other embodiments, the slug is delivered to the resin from about 1 to about 60 seconds, e.g., from about 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 57, 58, or 59 seconds. In certain other embodiments, the slug is delivered to the resin within seven seconds.

In other embodiments, the tight fluidic control and timing of the disclosed slug technique can allow the generation of over 100 unique reagent combinations to be delivered to the resin-bound growing peptide chain less than about 4 minutes, e.g., less than about 3 minutes, 2 minutes, 1.5 minutes, 60 seconds, 55 seconds, 50 seconds, 45 seconds, 40 seconds, 35 seconds, 30 seconds, 25 seconds, 20 seconds, 15 seconds, 10 seconds, 9 seconds, 8 seconds, 7 seconds, 6 seconds, 5 seconds, 4 seconds, 3 seconds, 2 seconds, or 1 second. In some embodiments, high-quality integrated data collection and process analytics can allow the utilization and integration of advanced machine learning models. In such embodiments, the disclosed methods and systems enable real-time proposal, outcome prediction, and the selection of candidate processing steps. In still other embodiments, the conducting of real-time process step selection can require the orchestration of asynchronous or semi-continuous events, as shown in FIG. 6. In such embodiments, advanced control techniques can necessitate the use of industrial process controllers for reliable and repeatable timing and coordination.

In some embodiments, slug flow formation requires asynchronous actions to create a slug for solid phase peptide synthesis (SPPS). In certain embodiments, the slug formation is asynchronous or semi-continuous, wherein the slugs are prepared asynchronously to the delivery of the reagents to the resin.

Peptide and protein synthesis requires changing between the coupling steps and deprotection steps. This typically requires long washout times when the system operates in the laminar flow regime. Laminar flow is known to have increased diffusion and dispersion in the system often requiring three residence times to obtain steady-state reaction conditions. Operating flow chemistry systems in this turbulent flow regime to get closer to the idealized plug flow regime is not a feasible solution due to the large amounts of solvent use resulting in an uneconomical process. Oscillating flow within a reactor can create vortices mimicking those created in a turbulent flow, which can increase mixing resulting in lower washout times and increased system productivity at lower flow rates.

As disclosed herein, the methods and systems further utilizes oscillatory flow within a continuous flow system containing a reactor with solid-supported resin for peptide or protein synthesis and/or cleavage. Under these conditions, highly turbulent mixing conditions are achieved without a stirring device or without high flowrates, thus, enhances the rate of a chemical reaction between liquid/liquid, liquid/gas, or liquid/solid phases while decreasing overall solvent use. Oscillating the flow stream creates turbulence resulting in higher mixing and reduced dispersion allowing for higher reaction control and reduced solvent utilization.

In other embodiments, the methods or systems described herein, are further subjected to oscillating flow within the microfluidic channel, thereby resulting in enhanced mixing and reaction kinetics. An oscillating flow occurs where the plurality of reagents is flowed back and forth between the inlet and outlet in the microfluidic channels to enhance sample utilization and synthetic sensitivity. In certain embodiments, oscillation is not used during slug flow. In other preferred embodiments, oscillation is used within the reactor. In certain preferred embodiments, oscillation is used during the peptide or protein cleaving step.

In some embodiments, the method or system further comprises subjecting to oscillation. In certain embodiments, the oscillation decreases washout time. In other embodiments, the oscillation decreases mixing time. In certain other embodiments, the oscillation decreases the coupling time of the protected amino acid or peptide to the unprotected amino acid or peptide that is immobilized on the resin.

In certain embodiments, utilizing oscillation decreases washout time, mixing time, and coupling time of the protected amino acid or peptide to the unprotected amino acid or peptide that is immobilized on the resin by more than about 5%, more than about 10%, more than about 20%, more than about 30%, more than about 40%, more than about 50%, more than about 60%, more than about 70%, more than about 80%, more than about 90%, more than about 95%, or more than about 99% then without oscillation.

In other embodiments, the temperature throughout the peptide or protein production is in the range of about −50° C. to about 295° C. In some embodiments, the temperature comprises the fluidic component temperature. In certain embodiments, the temperature comprises the resin temperature. In still other embodiments, the temperature is the slug temperature. In certain other embodiments, the reaction temperature is about −50° C. to about 295° C.

In some embodiments, the methods and systems described herein, deliver a plurality of reagents to a resin by semi-continuous or continuous manufacturing, wherein the reagents comprise: a) a protected amino acid or peptide; b) an activating agent; and c) a solvent, thereby coupling the protected amino acid or peptide to an unprotected amino acid, peptide or protein that is immobilized on the resin.

In other embodiments, the reagents further comprise a base. In some embodiments, the reagents further comprise a second activating agent. In certain embodiments, the reagents further comprise a chaotropic agent. A chaotropic agent is a denaturant, namely a compound that can disrupt the hydrogen bonding network between water molecules and reduce the stability of the native state of macromolecules, e.g. peptides and proteins in the present disclosure, by weakening the hydrophobic effect. In still other embodiments, the reagents further comprise an additive. In certain other embodiments, the reagents further comprise a wash solvent. In certain preferred embodiments, the reagents further comprise a deprotection agent, thereby deprotecting the peptide or protein that is immobilized on the resin, and optionally elongating the peptide or protein by coupling one or more amino acids or peptides sequentially to the peptide or protein that is immobilized on the resin.

In certain embodiments, amino acid or peptide activation is carried out in DMF as a solvent, i.e. the amino acid or peptide derivative, a coupling reagent and optionally an additive are dissolved in DMF and mixed. HATU or HBTU may be used as coupling reagent, although other coupling reagents, such as, TBTU or DEPBT may also be used to convert the Fmoc amino acid into an active OBt or ODhbt ester in the presence of a base, preferably DIPEA. The amino acid derivative of choice is pre-activated by incubation with the above reagents for 1 sec-30 min, e.g., for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 57, 58, 59, or 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 min before addition to the resin. The coupling reaction is allowed to proceed for 1 sec to 90 min, e.g., for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 57, 58, 59, or 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 97, 98, 99, or 90 min. The amino acid derivative may be used in a 0.01-10 molar ratio relative to the amount of resin-bound amine groups, e.g., at a molar ratio of 0.01, 0.02, 0.03, 0.04, 0.05, . . . 0.1, . . . 0.2, . . . 0.3, . . . 0.4, . . . 0.5, . . . 0.6, . . . 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, . . . 9.3, 9.4, 9.5, 9.6, 9.7, 9.8, 9.9, or 10. In order to achieve complete coupling, it may be advantageous to add a second activating agent or base to the reaction mixture after some time, e.g., after 10, 20, 30, 40, or 60 min. The pre-activation and coupling steps are commonly carried out at room temperature, but may also be performed at other temperatures. It may be advantageous to perform one or more re-coupling steps in order to achieve near to complete conversion of amino groups. In certain other embodiments, amino acid and peptide activation is carried out in a solvent comprising of NMP, dimethyl sulfoxide, dichloromethane, tetrahydrofuran, acetonitrile, toluene, DMF, or a combination thereof.

In still other embodiments, coupling agents include carbodiimide derivatives, for example N,N′-dicyclohexylcarbodiimide or N,N-diisopropylcarbodiimide. In certain other embodiments, coupling agents also include uronium or phosphonium salt derivatives of benzotriazol. Examples of such uronium and phosphonium salts include HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), and HAPyU (O-(benzotriazol-1-yl)oxybis-(pyrrolidino)-uronium hexafluorophosphate. In certain embodiments, the coupling reagent is HBTU, HATU, BOP, or PyBOP.

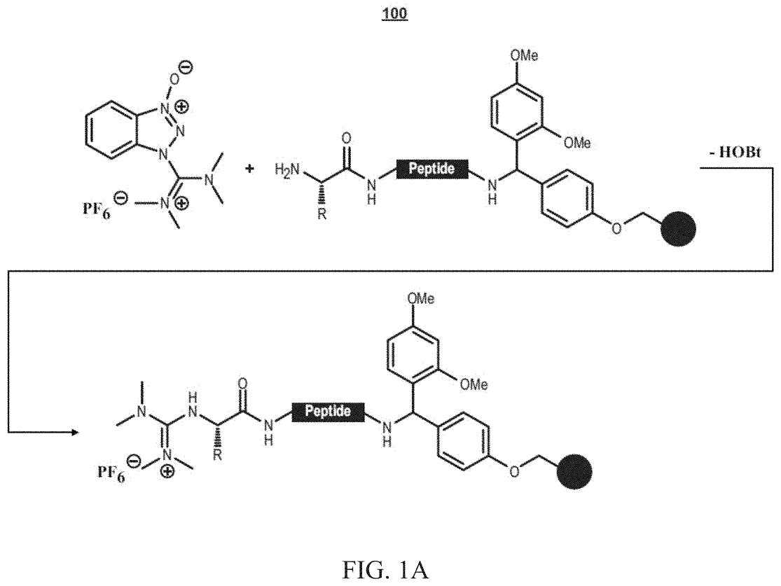

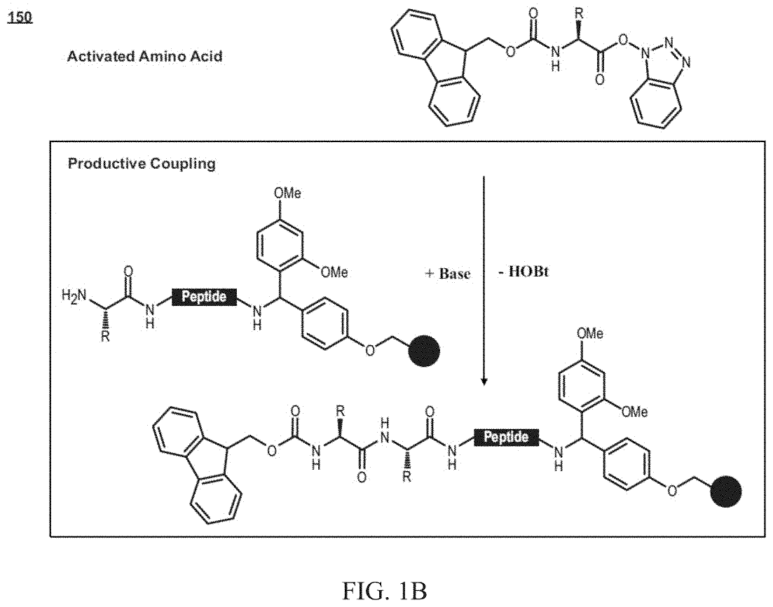

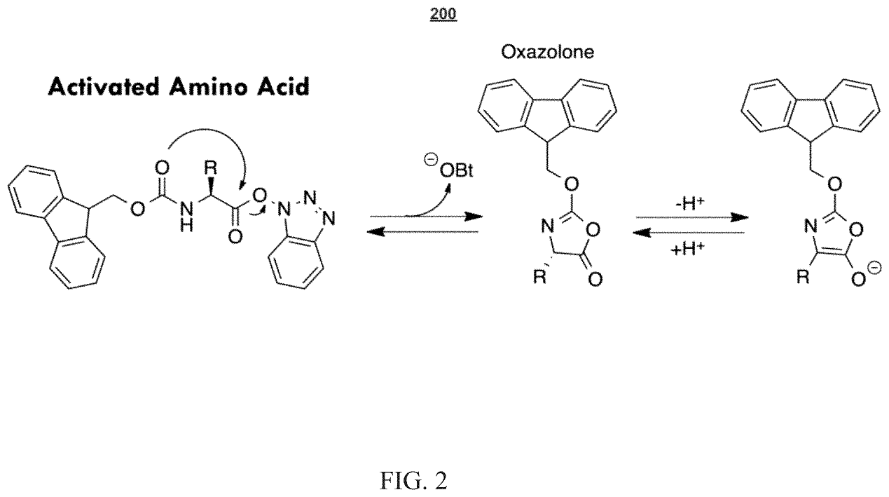

In some embodiments, the activating agent comprises HATU, HBTU, TBTU, DEPBT, PyAOP, PyBOP, DIC, DCC, COMU, HOBt, OBt ester, ODhbt ester, or a combination thereof. In other embodiments, the reagents further comprise a second activating agent. In certain embodiments, slug flow is further used to deliver a slug comprising a second activating agent. In preferred embodiments, the slug flow method and system maximizes mixing thereby reducing the effect of guanidinylation of low cost uronium activating agents that are not fully reacted prior to being delivered across the resin. Schemes 100, 150 and 200, FIGS. 1A, 1B and 2. In certain other embodiments, the deactivation of the activating agent is preferred prior to passing through the resin. Scheme 200, FIG. 2.

In other embodiments, the solvent comprises dimethyl formamide (DMF), N-methylpyrrolidone (NMP), dimethyl sulfoxide (DMSO) and dichloromethane (DCM), acetonitrile, water, methanol, ethanol and isopropanol (2-propanol), n-propanol, n-butanol, isobutanol, sec-butanol, tert-butanol, amyl alcohol, heptanol, octanol, nonanol, decanol, hexanol, dioxane, tetrahydrofuran, diglyme, or a combination thereof.

In some embodiments, the base comprises piperidine, 4-methyl piperidine, DBU, piperazine, morpholine, DIEA, DBN, 2,6 di-tert-butylpyridine, phasphazene bases, or a combination thereof.

In other embodiments, the wash solvent comprises DMA, DMF, NMP, dimethyl sulfoxide, dichloromethane, tetrahydrofuran, acetonitrile, toluene, HFIP, TFE, or a combination thereof.

In some embodiments, the chaotropic agent comprises n-butanol, ethanol, guanidinium chloride, lithium perchlorate, lithium acetate, magnesium chloride, phenol, 2-propanol, sodium dodecyl sulfate, thiourea, urea, trifluoroethanol, lithium bromide, DMSO, potassium thiocyanate, Triton X-100, or a combination thereof.

In certain embodiments, the additive comprises formic acid, trifluoromethanesulfonic (TFMSA), trifluoroacetic acid (TFA), trichloroacetic acid, p-toluenesulfonic acid (PTSA), dichloroacetic acid, taurine, chloroacetic acid, formic acid, 2,4-dinitrophenol, ascorbic acid, benzoic acid, hydroxybenzotriazole (HOBt), acetic acid, 4-nitrophenol, hexafluoroisopropanol (HFIP), phenol, benzenesulfonamide, piperidine, or a combination thereof.

The person skilled in the art would be well-aware of SPPS methods based on an Fmoc synthesis protocol. Each slug of amino acid addition to the resin typically starts with Fmoc cleavage, i.e. removal of the Fmoc protecting group from the resin-bound peptide chain. This is achieved by incubating the peptide resin with a base in a solvent capable of swelling the resin and dissolving the reagents. In some embodiments, slug flow is further used to deliver a slug comprising a base. Popular bases for this purpose comprise, e.g., secondary amines such as piperidine and 4-methyl piperidine. In other embodiments, the base comprises piperidine, 4-methyl piperidine, DBU, piperazine, morpholine, DIEA, DBN, 2,6 di-tert-butylpyridine, phasphazene bases, or mixtures thereof. Suitable solvents comprise, e.g., DMA, DMF, NMP, dimethyl sulfoxide, dichloromethane, tetrahydrofuran, acetonitrile, toluene, HFIP, TFE and mixtures thereof. The reaction is commonly carried out at ambient temperature, e.g., within a temperature range of 15-120° C. In certain embodiments, the base-labile and acid-stable Fmoc is split off by a short treatment (1 second to 10 minutes, e.g., for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, . . . 57, 58, 59, or 60 second, 1, 2, 3, 4, 5, 6, 7, 8, 9 or 10 minutes) with 5-50%, preferably 20%, piperidine in DMF (v/v). In still other embodiments, the treatment is repeated and/or slightly prolonged (5 second to 30 minutes, e.g., for 5, 6, 7, 8, 9, 10, 11, 12, 13, 14, 15, 16, 17, 18, 19, 20, 21, 22, 23, 24, 25, 26, 27, 28, 29, 30, . . . 57, 58, 59, or 60 second, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 minutes). For synthesis of large peptides with difficult-to-cleave stretches, the duration of Fmoc cleavage as well as the number of repetitions may be gradually increased. For instance, the cleavage time may be 15-75 minutes, e.g., 15, 30, 45, 60, or 75 minutes, and the cleavage may be repeated up to 8 times, e.g., 2, 3, 4, 5, 6, 7, or 8 times. Moreover, the temperature may be increased, e.g., to a temperature between 30° C. and 90° C. Under those conditions, complete deblocking is achieved in most cases. Alternatively, the reagent used for Fmoc cleavage may be varied. It is understood that the coupling of an amino acid derivative to the peptide resin, i.e. the lengthening step, is one of the central steps of the SPPS cycle.

As disclosed herein, the rate and yield of the method may be influenced by various parameters such as the choice of solvent, the steric hindrance, and the reactivity of the activated carboxylic acid. The solvent may not only determine the swelling of the precursor peptide-resin and may thus influence the accessibility of the reactive sites, e.g., it may directly affect the kinetics of the coupling reaction. Suitable solvents are capable of swelling the resin and dissolving the reagents and comprise, e.g., water, DMA, DMF, NMP, dimethyl sulfoxide, dichloromethane, tetrahydrofuran, acetonitrile, toluene, HFIP, TFE and mixtures thereof. The steric hindrance is determined by the nature of the amino acid side chains and their protecting groups. The reactivity of the activated carboxylic acid determines the acylation rate, as well as the extent of side reactions, such as racemization. Depending on the synthesis strategy chosen, peptide derivatives, such as, pseudoproline dipeptide derivatives, di- or tripeptide derivatives, or branched dipeptide derivatives may be used in lieu of single amino acid derivatives.

In certain embodiments, method and system is further used to deliver a slug comprising deprotection agents. It is well known that even slight variations of the agent may considerably accelerate the cleavage, e.g., the use of: 1 to 5% DBU in DMF, 20% piperidine or piperazine and 1-5% DBU in DMF, 20% piperidine or piperazine in NMP, or 20% piperidine or piperazine in DMF at 45° C., or up to 100° C. Certain mild cleavage conditions comprise of, e.g., 0.1 M HOBt plus 20% piperidine in DMF, 50% morpholine in DMF, 2% HOBt plus 2% hexamethyleneimine plus 25% N-methylpyrrolidine in 50% DMSO in NMP. In some embodiments, the Fmoc protecting group is cleaved off the growing peptide chain that is conjugated to the solid phase using a mixture comprising of 5-50% (v/v) piperidine or 4-methyl piperidine in N,N-dimethylformamide (DMF), 5-50% (v/v) piperidine or 4-methyl piperidine in N-methylpyrrolidone (NMP), 1-5% (v/v) diazabicyclo[5.4.0]undec-7-ene (DBU) in DMF, and 50% (v/v) morpholine in DMF. In certain other embodiments, the deprotection agent comprises at least about 50% (v/v) TFA/water; at least about 75% (v/v) TFA/water; at least about 80% (v/v) TFA/water); at least about 90% (v/v) TFA/water; at least about 1% to about 5% DBU in DMF; at least about 20% piperidine or piperazine and at least about 1.5% to about 5% DBU in DMF; at least about 20% piperidine or piperazine in NMP; at least about 20% piperidine or piperazine in DMF; at least about 0.1 M HOBt and about 20% piperidine in DMF, at least about 50% morpholine in DMF, at least about 2% HOBt and about 2% hexamethyleneimine and about 25% N-methylpyrrolidine in about 50% DMSO in NMP; at least about 5% to about 50% (v/v) piperidine or 4-methyl piperidine in N,N-dimethylformamide (DMF); at least about 5% to about 50% (v/v) piperidine or 4-methyl piperidine in N-methylpyrrolidone (NMP); at least about 1% to about 5% (v/v) diazabicyclo[5.4.0]undec-7-ene (DBU) in DMF; at least about 50% (v/v) morpholine in DMF, or a combination thereof.

In some embodiments, the resin comprises polystyrene, polystyrene-PEG composites, PEG, PEGA, cross-linked ethoxylate acrylate (CLEAR), polyamides, polydimethylacrylamide, 4-alkoxybenzyl alcohol (Wang) resin, diphenyldiazomethane (PDDM) resin, 4-(2′,4′-dimethoxyphenyl-Fmoc-aminomethyl)-phenoxymethyl-polystyrene (Rink) resin, 2-methoxy-4-alkoxybenzyl alcohol (Sasrin) resin, or 2-Chlorotrityl chloride (CTC) resin.

In other embodiments, the method or system for producing a peptide or protein further comprises fluidic components to mix and meter reagents.

In certain embodiments, the reaction conditions can be monitored to assess the efficiency of the reaction conditions by evaluating the concentration of the peptide or protein synthesis reaction. In other embodiments, monitoring of the concentration of the reaction can be accomplished inside the system, wherein the solute can be, for example, an unreacted amino acid or a protecting agent. Monitoring the concentration can be achieved using an apparatus comprising Raman spectroscopy, ultraviolet spectroscopy, infrared spectroscopy, circular dichroism spectroscopy, liquid chromatography, high performance liquid chromatography, mass spectrometry, conductivity, or a combination thereof.

In some embodiments, the method or systems described herein, comprises reaction conditions comprising: a) concentrations; b) residence time; c) mixing time; d) temperature; e) flowrate; or a combination thereof. In other embodiments, the concentrations comprise a plurality of reagents. In certain other embodiments, the concentrations further comprise reaction byproducts. In still other embodiments, the reaction byproducts are measured. In certain embodiments, the reaction byproducts are measured after the outlet of the resin.

In certain embodiments, the reaction byproducts are measured within the slug. In other embodiments, the reaction byproducts are measured after the outlet of the resin.

In other embodiments, the residence time is calculated using step input of an amino acid tracer, allowing the monitoring of the unreacted protected amino acid or peptide concentration within the reaction solvent at the outlet of the resin. In some embodiments, the protected reacted amino acid or peptide is used as the reference. In certain embodiments, the temperature comprises the fluidic component temperature. In still other embodiments, the temperature comprises the resin temperature. In some embodiments, the temperature is about −50° C. to about 295° C. In certain embodiments, the temperature is about 30° C. to about 90° C., e.g., about 35° C., 40° C., 45° C., 50° C., 55° C., 60° C., 65° C., 70° C., 75° C., 80° C., 85° C. to about 90° C.

In some embodiments, the flowrate is about 1.0 μL/min to about 10 L/min, e.g., about 2 mL/min to about 10 mL/min, about 40 mL/min, or about 80 mL/min. In certain embodiments, the flowrate is about 1 L/min to about 10 L/min.

During peptide or protein synthesis, it is essential to prevent the formation of secondary and tertiary structures and peptide-peptide interactions which can cause the reaction rate to be reduced. In some embodiments, the ionic property of the peptides or proteins can allow a static or low frequency high voltage electric field to effectively disrupt the formation of secondary and tertiary structures and peptide-peptide interactions in growing peptides, such as, alpha and beta sheet formation. In other embodiments, the utilization of low frequency and static electric fields can prevent dielectric heating of the reaction mixture, which prevents the peptide chain from growing. As used herein, the term “dielectric heating” refers to heating via the application of an alternating electric field (referred to herein as a “dielectric field”), preferably in the wavelengths between approximately 1500 centimeters to 0.3 gigameters. In certain embodiments as described herein, the methods and systems for producing a peptide or protein using slug flow to deliver a slug to a resin uses an electric field. In other embodiments, the resin is contained inside of an electric field reaction vessel as described in U.S. Application No. 62/839,392, the contents of which are incorporated herein by reference in its entirety for all purposes. In such embodiments, the electric field reduces peptide or protein aggregation.

In other embodiments, the methods and systems for producing a peptide or protein using slug flow to deliver a slug to a resin uses an electric field, wherein the electric field reduces peptide or protein aggregation.

In certain embodiments, the methods and systems described herein, leverages automated flow technology and inline analytics to optimize the production of peptides and proteins by delivering a plurality of reagents to a resin by semi-continuous or continuous manufacturing. In some embodiments, the methods and systems uses automation and inline analytics to enable up to 50% increase in synthetic efficiency compared to standard SPPS manufacturing practices. In certain embodiments, the combination of the methods and systems described herein, with an automated system and inline analytics can achieve longer length peptides and proteins more rapidly and efficiently. In such embodiments, the slug lengths are easily controlled resulting in high mass transfer thereby improving product production.

In some embodiments, the method and system comprises inline analytics capable of making in-sequence optimization. In other embodiments, the inline analytics optimize the delivery of amino acids or peptides to lengthen the unprotected amino acid, peptide or protein that is immobilized on the resin.

In other embodiments, the inline analytics comprise: a) measuring reaction conditions; b) measuring reaction yields; c) measuring the effectiveness of the solid phase peptide synthesis (SPPS); d) measuring the temperature; or a combination thereof. In some embodiments, the reaction conditions comprise: a) concentrations; b) residence time; c) mixing time; d) temperature; and e) flowrate. In certain embodiments, the concentrations comprise a plurality of reagents, unreacted reagents and reaction byproducts. In still other embodiments, the concentrations comprise reagents. In certain other embodiments, the concentrations comprise reaction byproducts.

In some embodiments, the method or system comprises producing a first and a second signal at a detection zone positioned downstream of the resin, comparing the first signal to the second signal and/or to a reference signal, and modulating a parameter of the system prior to and/or during a reaction at the resin. The modulating is based at least in part on information derived from the comparing step. The modulated parameters comprise any of: a flow rate, a reaction time, a temperature, a reactant type, a reactant concentration, a ratio of reactants, an addition of an additive, or combinations thereof.

The methods and systems described herein, have the ability to measure reaction yield and optimization utilizing inline process analytical technologies (PAT), allowing near real-time feedback on the effectiveness of the slug flow techniques to enable controlled delivery of amino acids and peptides during synthesis. In some embodiments, the PAT works by measuring the protecting group, such as, Fmoc, produced during the deprotection step, giving a near quantitative measurement of the coupling yield which allows the extraction of information about the coupling efficiency at every step of the synthesis process, enhancing the ability to model and probe the effect of the slug flow technique in near real-time. The information can be correlated with liquid chromatograph mass spectrometry (LC-MS) data to give a full understanding of any issue that can occur during the synthesis process. In some embodiments, the system further comprises inline analytics capable of making in-sequence optimization. In certain embodiments, the production is automated to lengthen the peptide chain. In other embodiments, the software developed for the apparatus can communicate with an e-commerce platform to automatically manufacture clients submitted peptides. In still other embodiments, the methods and apparatus automatically pushes the synthesis data from the PAT to a database. A PAT tool may be any tool that fits under the following categories: (1) acquire/analyze data (multivariate capable); (2) processing analyzer; (3) process control tool; and (4) management tool that allow for continuous improvement and knowledge of a process. Multivariate tools may be statistical designs of experiments (DOE). Combining these DOE studies with some type of computer software (as a process control tool) to control and alter processing conditions would fit under this category. In this case, a predictive equation or results from a DOE study may then be used to adjust the final formulation when process variations are encountered. Process analyzing tools can be implemented in three ways: (1) at-line, or where a sample is removed and isolated from a system; (2) on-line, or where a sample is diverted, measured and returned to the process; or (3) in-line, or where a sample is measured directly in the process. Process analyzers generate large amounts of data that can be collected and stored for quality control purposes and reporting. Lastly, analysis of data to build on the understanding of the overall process will aid in a continuous learning and improvement of the processing stream, which will facilitate regulatory acceptance and provide evidence to support alterations to an existing process.

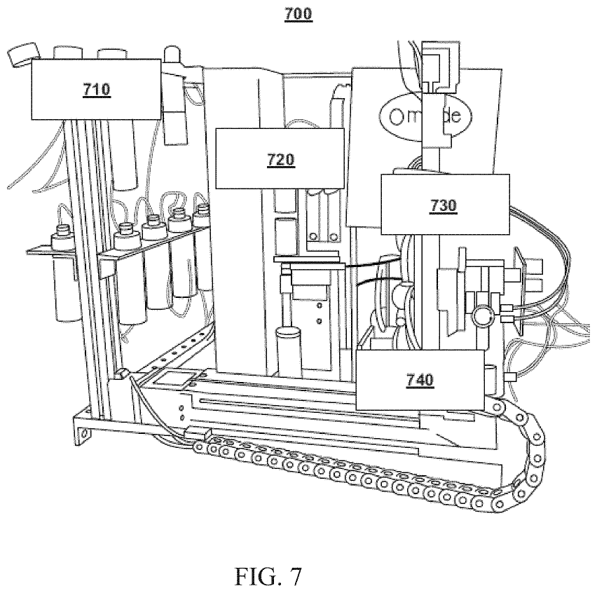

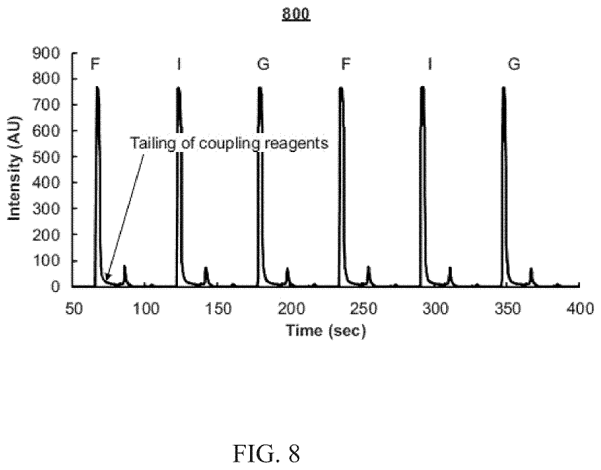

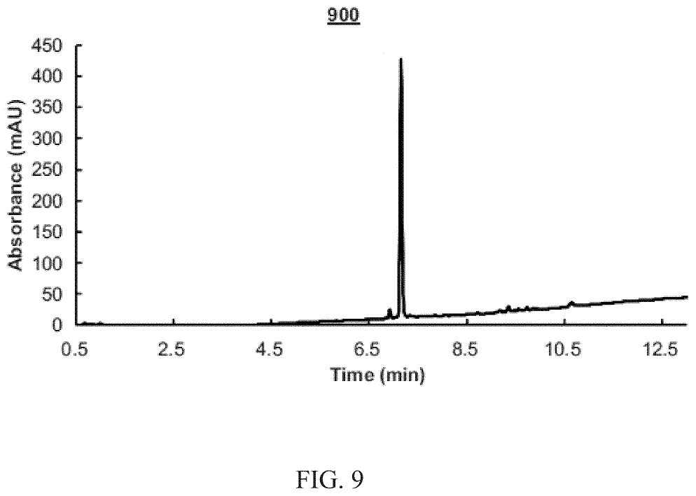

In other embodiments, the methods and systems are integrated into a bench top apparatus 700 that move the reagents 710 utilizing continuous solid phase peptide synthesis, capable of carrying out 10 syntheses with high yield at 2 minutes per coupling cycle. FIG. 7. The system utilizes fluidic components, such as an automated reactor 720 and reaction vessels 730, to accurately mix and meter all reagents. In addition, a robotic arm 740 allows automated synthesis of up to 100 peptides before needing to be reloaded with new reaction vessels. In some embodiments, the software developed for the apparatus can communicate with an e-commerce platform to automatically manufacture clients submitted peptides. In still other embodiments, the methods and apparatus automatically pushes the synthesis data from the PAT to a database. In certain other embodiments, system 700 has an integrated UV detector capable of capturing data at the outlet of the reactor. FIGS. 8, 9 and 10. In certain embodiments, system 700 has an integrated UV detector capable of capturing data 800 at the outlet of the reactor, e.g., no slug flow, 254 nm (FIG. 8). In preferred embodiments, system 700 utilizes fluidic components to accurately mix and meter all reagents. In certain embodiments, system 700 can synthesize peptides with purities greater than about 80%, preferably greater than about 90% (FIG. 9). As shown in FIG. 9, the methods and systems disclosed herein, provides the formation of acyl carrier protein (ACP(65-74)) at a purity of greater than 92% (UV220). Experimentally validated antimicrobial peptides, e.g., E16LKL, T7 Novispirin, Myxinidin (Y8), WMD4R, C18AA, Pexiganin, Adepantin-1, Pint, SHc-CATH, and LL-37 Pentamide can also be efficiently produced using the methods and systems described herein.

In some embodiments, the progress of the SPPS reaction may be monitored using inline analytics to ensure efficient Fmoc removal and/or the coupling steps. Fmoc determination on one hand and determination of free amines on the other hand may result in complementary information. Taken together, these methods may enable efficient monitoring of each step of the SPPS process. In other embodiments, the disclosure has the flexibility to be able to do PAT at each step. Some of the inline analytics usable in the context of the present disclosure are exemplified below.

In certain embodiments, the amount of Fmoc cleaved from the resin-bound peptide may easily be quantified by inline analytics. The Fmoc cleavage reagent drained from the resin may be monitored and the Fmoc concentration therein determined, e.g., by measuring the absorbance. Based on the amount of Fmoc cleaved off, the resin load, i.e. the original amount of Fmoc peptide on the resin, may be calculated. Further, to assess the completeness of Fmoc removal, a small sample of presumably Fmoc-deprotected resin may be subjected to an additional Fmoc cleavage protocol in order to determine the amount of residual Fmoc removed by the treatment. In other embodiments, a small scale test cleavage of the peptide or protein from a resin sample may be carried out in order to assess the completeness of Fmoc removal, efficiency of the coupling step and side product formation. The resulting peptides or proteins may be analyzed by analytical reversed phase-high performance liquid chromatography (RP-HPLC) using a standard gradient, where Fmoc protected and free peptide or protein sequences are separated. In still other embodiments, the peptide or protein sample may be analyzed by mass spectrometry, e.g., by LC-MS or matrix-assisted laser desorption ionization mass spectrometry (MALDI-MS). In certain other embodiments, the amount of free amines on the resin may be assessed by various assays, including the colorimetric Kaiser (i.e. Ninhydrin), TNBS, Chloranil, and Bromophenol Blue tests, or other methods known to the skilled artisan.

In other embodiments, the production is automated to lengthen the peptide or protein chain that is immobilized on the resin. In some embodiments, the automation comprises a robotic arm. In certain embodiments, the inline analytics optimize the delivery of amino acids or peptides to lengthen the peptide or protein chain. In still other embodiments, the peptides comprise homo-oligopeptides.

In certain embodiments, the methods and systems described herein, can be performed to optimize peptide and protein synthesis efficiency by monitoring the reaction quantities or yields. As peptides remain aggregated, the steric hindrance effects the rate of reaction and therefore the rate at which products are formed and starting materials are consumed. As described herein, the reaction efficiency can be assessed through monitoring reaction yields using inline analytical technologies. The inline analytics work, for example, by monitoring reaction products and reagents, which results in near quantitative measurement of the coupling yield to provide near real-time feedback on the effectiveness of the slug flow during peptide or protein synthesis. In some embodiments, reaction products or reagents are detected at or near the reactor outlet, or at any inline position after the reactor outlet. In other embodiments, the reaction products or reagents are detected or monitored proximal to the reactor outlet. In still other embodiments, reaction products or reagents that are detected or monitored include, for example, free amino acids that remain unbound to the nascent peptide chains, or Fmoc that is produced during the deprotection step. In certain other embodiments, the reaction yields relative to the peptide length is measured in about near real-time at the outlet before the next slug is added to the system.

In some embodiments, the method or system produces a coupling efficiency of at greater than about 90%, about 95%, about 98%, about 99%, or about 99.5%. In other embodiments, the coupling is repeated. In certain embodiments, the coupling is automated. In still other embodiments, the methods and systems are capable of completing a coupling step every 30 seconds, allowing for up to 2,880 couplings per day.

Where a peptide or protein has been synthesized by SPPS, acylation may be carried out prior to or after cleavage from the resin. In some embodiments, the method or system comprises acylating the N-terminal amino group prior to cleaving the peptide, e.g., endcapping groups. Acylation of the N-amino group of an amino acid may be carried out by reacting an amino acid or peptide with an acylating agent in the presence of base, e.g., diisopropylethylamine, in a suitable solvent, e.g., DMF. Non-limiting examples of acylating agents include acid halides, e.g., acid chlorides such as acetyl chloride, and acid anhydrides, e.g., acetic anhydride. Non-limiting examples of suitable bases include triethylamine, diisopropylethylamine, 4-methylmorpholine, and the like. In certain embodiments, small changes to the molecular weight of the peptide or protein, or changes to the endcapping groups, e.g., acyl groups, of the peptide or protein, while leaving the main chain, e.g., backbone, of the peptide or protein the same, can enhance the overall synthesis of the target peptide or protein.

In some embodiments, the endcapping can be performed for less than about 5 minutes. In other embodiments, the endcapping can be performed for less than about 1 minutes. In certain embodiments, the endcapping can be performed for less than about 30 seconds. In preferred embodiments, the endcapping can be performed for less than about 10 seconds.

In other embodiments, the endcapping protocol can be performed at room temperature. In some embodiments, the endcapping protocol can be performed in a temperature range of about −50° C. to about 295° C.

The solvent used in the endcapping protocol is preferably a polar non-aqueous solvent, e.g., acetonitrile, dimethyl sulfoxide (DMSO), methanol, methylene chloride, N,N-dimethylacetamide (DMA), N,N-dimethylformamide (DMF), N-methylpyrrolidone, or a combination thereof. In preferred embodiments, the solvent used in the endcapping protocol is DMF.

After the desired amino acid sequence has been synthesized, the peptide is cleaved from the resin. The conditions used in this process depend on the sensitivity of the amino acid composition of the peptide and the side-chain protecting groups. Generally, cleavage is carried out in an environment containing a plurality of scavenging agents to quench the reactive carbonium ions that originate from the protective groups and linkers. Common cleaving agents include, but are not limited to TFA and hydrogen fluoride (HF). In some embodiments, where the peptide is bound to the solid phase support via a linker, the peptide chain is cleaved from the solid phase support by cleaving the peptide from the linker. The conditions used for cleaving the peptide from the resin may concomitantly remove one or more side-chain protecting groups.

For cleaving the peptide or protein from the resin, any suitable composition known in the art may be used. The process may be conducted by using a composition comprising greater than about 50% (v/v) TFA, preferably more than about 75% (v/v) TFA, in particular at least about 80% (v/v) or even at least about 90% (v/v) TFA. In some embodiments, the composition may also comprise water and/or one or more scavengers, preferably, TFA, water and one or more scavengers. Particularly advantageous scavengers are thiol scavengers such as EDT and/or silane scavengers such as, e.g., TIPS. In other embodiments, the cleavage composition may comprise at least about 80% TFA, preferably at least about 90% TFA, and EDT. In still other embodiments, the cleavage composition may comprise at least about 80% TFA, preferably at least about 90% TFA, water, and EDT. In certain other embodiments, the cleavage composition may comprise at least about 80% TFA, preferably at least about 90% TFA, water, and TIPS. In certain embodiments, the cleavage composition may comprise at least about 80% TFA, preferably at least about 90% TFA, water, TIPS and EDT. Exemplary, compositions for use in the context of the present disclosure may comprise of TFA/water/TIPS (90:5:5) v/v/v, TFA/water/phenol (90:5:5) v/v/v, TFA/water/EDT/TIPS (90:5:2.5:2.5) v/v/v/v, TFA/water/EDT/TIPS (90:4:3:3) v/v/v/v, TFA/water/EDT (90:5:5) v/v/v, TFA/thioanisole/anisole/EDT (90:5:3:2) v/v/v/v, TFA/thioanisole/water/phenol/EDT (82.5:5:5:5:2.5) v/v/v/v/v.

In certain embodiments, the step of cleaving the precursor peptide or protein off the resin may be carried out under any conditions described herein. Cleavage can be carried out by incubating the washed resin with the cleavage composition for about 1 to about 4 h and/or at a temperature of about 0 to about 32° C. Exemplarily, cleavage may be carried out by incubating the washed resin with the cleavage composition for up to about 1 h, up to about 1.5 h, up to about 2 h, up to about 2.5 h, up to about 3 h, up to about 3.5 h or up to about 4 h or longer than 4 h at a temperature of about 0 to about 4° C., about 4 to about 10° C., about 10 to about 15° C., about 15 to about 25° C., or about 25 to about 100° C. Exemplarily, cleavage may be carried out by incubating the washed resin with the cleavage composition for up to about 1 h, up to about 1.5 h, up to about 2 h, up to about 2.5 h, up to about 3 h, up to about 3.5 h or up to about 4 h or longer than 4 h at a temperature of about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32° C. For example, the cleavage of protected peptide fragments from 2-chlorotrityl resin may be achieved using TFE/AcOH/DCM (1:1:3), 0.5% TFA/DCM, or HFIP/DCM.

In other embodiments, the peptide or protein that is immobilized on the resin is cleaved from the resin after deprotection. In some embodiments, the peptide or protein that is immobilized on the resin is cleaved from the resin prior to deprotection.

The methods and systems described herein, further uses a linker to isolate the nontarget peptides or proteins from the crude reaction solutions prior to liquid chromatograph (LC) purification of the target peptide or protein. In some embodiments, a linker comprising an azide is coupled to the unprotected N-terminus of the growing amino acid, peptide or protein chain that is that is immobilized on the resin. In other embodiments, the linker is coupled to the unprotected N-terminus between the protected amino acid or peptide coupling step. Upon completion of the synthesis of the target peptide or protein, the target peptide or protein is cleaved from the resin and the resulting reaction mixture is subjected to a click resin to remove the nontarget peptides or proteins allowing ease of purification of the target peptide or protein. Thus, the disclosed purification technique allows efficient removal of nontarget peptides or proteins from the crude reaction solutions resulting in less impurities prior to purification of the crude target peptide or protein by size-exclusion chromatography (SEC), strong cation exchange (SCX) chromatography, reversed-phase (RP) chromatography, or other standard chromatography methods known in the art.

In certain embodiments, the method or system produces peptides from between about 5 to about 100 amino acids in length. In other embodiments, the method or system produces proteins from between about 101 to about 1000 amino acids in length. In still other embodiments, the method or system for producing a peptide or protein comprises liquid or solid phase peptide synthesis, preferably solid phase peptide synthesis. In certain other embodiments, the method or system for producing a peptide or protein further comprises fluidic components to mix and meter reagents

In certain other embodiments, the peptides can be from about 5 to about 10 amino acids, from about 10 to about 20 amino acids, from about 20 to about 30 amino acids, from about 30 to about 40 amino acids, from about 40 to about 50 amino acids, from about 50 to about 60 amino acids, from about 60 to about 70 amino acids, from about 70 to about 80 amino acids, from about 80 to about 90 amino acids, from about 90 to about 100 amino acids, or more than 100 amino acids in length.

In some embodiments, the peptide produced is about 5 to about 100 amino acids in length. In such embodiments, the methods and systems described herein, are capable of producing a peptide of about 100 amino acids in length in about 82% yield at a rate of about 7 amino acids per second since byproduct formation can be fully suppressed. In other embodiments, the methods and systems are capable of producing a peptide of about 100 amino acids in length in less than about 14 days, e.g., about 13 days, about 12 days, about 11 days, about 10 days, about 9 days, about 8 days, about 7 days, about 6 days, about 5 days, about 4 days, about 3 days, about 2 days, about 1 day, about 20 hours, about 16 hours, about 12 hours, about 8 hours, or about 4 hours.

In other embodiments, the methods and systems are capable of forming a peptide of about 60 amino acids in length in less than about 4 days, about 3 days, about 2 days, about 1 day, about 20 hours, about 16 hours, about 12 hours, about 8 hours, or about 4 hours. In some embodiments, a 60 amino acid peptide is formed in less than about 4 days. In certain embodiments, a 60 amino acid peptide is formed in less than about 3 days. In still other embodiments, a 60 amino acid peptide is formed in less than about 2 days.

In some embodiments, the methods and systems are capable of forming a peptide of about 20 amino acids in length in less than about 2 days, about 1 day, about 20 hours, about 16 hours, about 12 hours, about 8 hours, or about 4 hours. In other embodiments, a 20 amino acid peptide is formed in less than about 24 hours. In certain embodiments, a 20 amino acid peptide is formed in less than about 8 hours.

In certain embodiments, the reaction yields relative to the peptide or protein length is measured in about real-time. In some embodiments, the reaction yields are a purity of at least about 70%, e.g., about 80%, about 90%, about 95%, or about 99% for a population of peptides from between about 5 to about 100 amino acids. In other embodiments, the reaction yields are a purity of at least about 70%, e.g., about 80%, about 90%, about 95%, or about 99% for a population of proteins from between about 101 to about 1000 residues in length. In still other embodiments, applying inline analytics to the synthesis reaction can produce a reaction yield purity at least about 30%, e.g., of at least about 40%, of at least about 50%, of at least about 60%, of at least about 70%, of at least about 80%, of at least about 85%, of at least about 90%, of at least about 91%, of at least about 92%, of at least about 93%, of at least about 94%, of at least about 95%, of at least about 96%, of at least about 97%, of at least about 98%, of at least about 99% for a population of peptides from between about 2 to about 20, about 2 to about 30, and about 30 to about 100 amino acids or a population of proteins from between about 101 to about 1000 residues in length than a method or system without inline analytics.

In other embodiments, the peptide yield is relative to the peptide length and is measured in about real-time at the outlet of the resin.

In some embodiments, the method or system can produce a 50 amino acid desalted peptide at a purity of >80% within 3 days. In other embodiments, the method or system can produce a 20 amino acid desalted peptide at a purity of >70% within 24 hours. In certain embodiments, the method or system can produce a 20 amino acid desalted peptide at a purity of >90% within 8 hours, preferably within 4 hours. In still other embodiments, the slug flow technique allows for the synthesis of peptides within the 40-100 amino acid length. In certain other embodiments, slug flow does not require high temperatures or modified reaction conditions which may cause racemization of amino acids and coupling difficulties.

In other embodiments, the peptide yield is a purity of at least about 70%, about 80%, about 90%, about 95%, or about 99% for a peptide from about 5 to about 100 amino acids in length. In some embodiments, the 60 amino acid peptide is formed with a purity of greater than about 80%. In certain embodiments, the 60 amino acid peptide is formed with a purity of greater than about 90%. In still other embodiments, the 20 amino acid peptide is formed with a purity of greater than about 85%. In certain other embodiments, the 20 amino acid peptide is formed with a purity of greater than about 95%.

In some embodiments, the protein produced is about 101 to about 1000 amino acids in length. In other embodiments, the methods and systems are capable of producing a protein in less than about 14 days, e.g., about 13 days, about 12 days, about 11 days, about 10 days, about 9 days, about 8 days, about 7 days, about 6 days, about 5 days, about 4 days, about 3 days, about 2 days, about 1 day, about 20 hours, about 16 hours, about 12 hours, about 8 hours, or about 4 hours.

In other embodiments, the protein yield is relative to the protein length and is measured in about real-time at the outlet of the resin. In certain other embodiments, slug flow does not require high temperatures or modified reaction conditions which may cause racemization of amino acids and coupling difficulties.

In some embodiments, the protein yield is a purity of at least about 70%, about 80%, about 90%, about 95%, or about 99% for a protein from about 101 to about 1000 amino acids in length. In other embodiments, the protein is formed with a purity of greater than about 80%. In certain embodiments, the protein is formed with a purity of greater than about 90%.

The person skilled in the art will routinely optimize the compositions for use in the context of the present disclosure depending on the amino acid composition of the peptide or protein in question and will envisage the optional use of one or more scavengers such as, inter alia, DTE, EDT, TES, TIPS, 2-mercaptoethanol, ethyl methyl sulfide, m- or p-cresol, 2-Me-indole, Ac-Trp-OMe, or tryptamine. In the context of the present disclosure, the term “scavengers” is used to refer to compounds which are added to the reaction mixture in order to suppress side reactions during cleavage of a peptide from the resin after SPPS and/or during removal of protecting groups. Typical scavengers used in a cleavage composition are “thiol scavengers” (e.g., EDT, DTE, DTT, and beta-mercaptoethanol) and “silane scavengers” (e.g., TES and TIPS). Further commonly used scavengers comprise ethyl methyl sulfide, thioanisole, anisole, m- or p-cresol, 2-Me-indole, Ac-Trp-OMe, or tryptamine.

In some embodiments, the scavengers may serve to prevent undesired side reactions with sensitive amino acids such as Cys, Met, Ser, Thr, Trp, and Tyr. Without wishing to be bound by any theory, it is believed that the side reactions are suppressed by capture of the highly reactive carbocations generated during the deprotection and/or cleavage process.

Filtration may be any filtration method known in the art, such as, e.g., dead-end filtration or cross-flow filtration. As used herein, the terms “cross-flow filtration”, “crossflow filtration”, “tangential flow filtration” or “tangential filtration” may be understood interchangeably. The filter may be of any material known in the context of filtration in the art, such as, e.g., plastic (e.g., nylon, polystyrene), metal, alloy, glass, ceramics, cellophane, cellulose, or composite material. The filter may be hydrophobic or hydrophilic. The surface of the filter may be neutral or positively charged or negatively charged.

The disclosure generically described, will be more readily understood by reference to the following examples, which are included merely for purposes of illustration of certain aspects and embodiments of the present disclosure, and are not intended to be limiting.

### Exemplification

### Abbreviations

aa amino acids
Ac acetyl
ACS aggregate causing sequences
Boc t-butyl carbamate
BME beta-mercaptoethanol
BSA bovine serum albumin
° C. degrees Celsius
cm centimeter
COMU 1-[(1-(Cyano-2-ethoxy-2-oxoethylideneaminooxy) dimethylaminomorpholino)] uronium
CTC 2-Chlorotrityl chloride
DBU 1,8-diazabicyclo[5.4.0]undec-7-ene
DBN 1,5-diazabicyclo[4.3.0]non-5-ene
DCC N,N′-dicyclohexylcarbodiimide
DCM dichloromethane
DEPBT 3(diethoxyphosphoryloxy)-1,2,3-benzotriazine-4(3H)-one
DIC N,N′-diisopropylcarbodiimide
DIPEA diisopropylethylamine
DMA dimethylacetamide
DMF dimethyl formamide
DMSO dimethyl sulfoxide
DTE dithioerythritol
DTT dithiothreitol
EDT 1,2-ethanedithiol
eq. equivalent
Et ethyl
ETFE polyethylenetetrafluoroethylene
Fmoc 9-fluorenylmethyl carbamate
h hour
HATU hexafluorophosphate azabenzotriazole tetramethyl uronium
HBTU 2-(1H-Benzotriazole-1-yl)-1,1,3,3-tetramethyluronium hexafluorophosphate
HFIP hexafluoroisopropanol
HOBt hydroxybenzotriazole
HODHBt 3-hydroxy-1,2,3-benzotriazin-4(3H)-one
HPLC high performance liquid chromatography
Hz hertz
kJ kilojoules
LB lysogeny broth
LC-MS liquid chromatograph mass spectrometry
m meta
MALDI-MS matrix-assisted laser desorption ionization mass spectrometry
Me methyl
MHA Mueller-Hinton agar
MHB Mueller Hinton broth
MHz megahertz
min minute
μL microliter
μg microgram
mL milliliter
mm millimeter
mM millimolar
mol mole
nm nanometer
NMP N-methylpyrrolidone
p para
PAT process analytical technologies
PDDM diphenyldiazomethane
PEG polyethylene glycol
PEGA polyethylene glycol polyacrylamide
PIP piperidine
ps picosecond
PTFE polytetrafluoroethylene
PVDF polyvinylidene fluoride
PyAOP 7-azabenzotriazol-1-yloxy)tripyrrolidinophosphonium hexafluorophosphate
PyBOP benzotriazol-1-yl-oxytripyrrolidinophosphonium hexafluorophosphate
RP-HPLC reversed phase-high performance liquid chromatography
SASA solvent accessible surface area
sec second
SHP soybean hydrophobic protein
SPPS solid phase peptide synthesis
t tertiary
TBTU 2(1-H-benzotriazole-1-yl)-1,1,3-tetramethyl-uronium tetrafluoroborate
tert tertiary
TES triethylsilyl
TFA trifluoroacetic acid
TFE 2,2,2-trifluoroethanol
TIPS triisopropylsilyl
TNBS 2,4,6-trinitrobenzene sulfonic acid
TSB trypticase soy broth
UHMW ultrahigh molecular weight polyethylene
UV ultraviolet
V volts

Exemplary Method and System

Slug Formation Steps:

1. Fill sample loops with amino acid and activator.

2. Stop flow in main flow path.

3. Switch sample loop valves to be inline with the main loop.

4. Introduce gas bubble into the main flow path.

5. Start amino acid, activator, and base, e.g., DIEA pumps to push fluids into the main flow stream to form a slug.

6. Switch sample loop valve to be inline with secondary fill loop.

7. Introduce gas bubble into the main flow path.

8. Start main flow path pumps.

9. Allow slug to be mixed before entering the reactor or remove gas bubble before it enters the reactor.

10. React the protected amino acid with substrate immobilized on the resin (amino acid, peptide or protein) in the reactor.

11. Wash reactor to remove unreacted amino acid from the reactor.

12. Introduce deprotection agent.

13. Wash reactor.

14. Repeat above steps.

15. Optionally cleave target peptide or protein from resin prior to final deprotection step.

16. Cleave target peptide or protein from resin after final deprotection step.

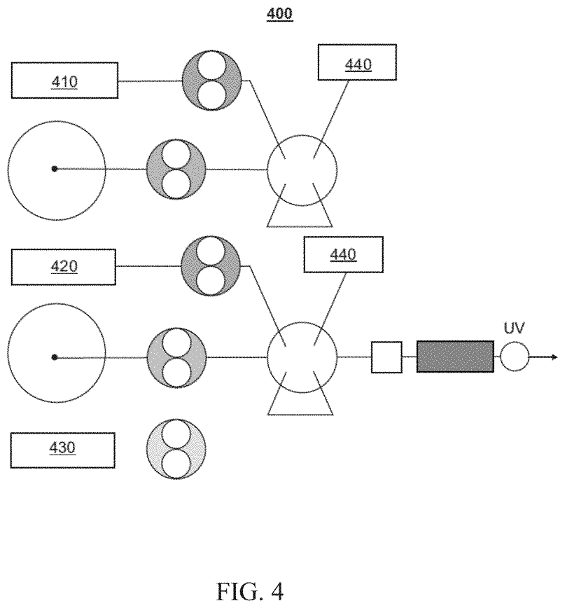

In some embodiments, the methods and systems for slug formation are shown in FIGS. 3-5. FIGS. 4 and 5 are schematic illustrations of exemplary systems 400 and 500 which can be used to perform slug flow processes described herein. The methods and systems described herein (system 400 in FIG. 4 and system 500 in FIG. 5) can involve, in other embodiments, slug flow synthesis, as opposed to batch based synthesis or semi-batch based synthesis, which are employed in many traditional solid phase peptide synthesis systems. In such embodiments, slug flow peptide or protein synthesis can be performed, in which fluid, of one form or another, is transported over the immobilized peptides or proteins 575.

In certain embodiments, reagents, e.g., amino acid 410, activator 420, and base 430, are transported throughout the system by slug flow. In certain other embodiments, reagents, e.g., amino acids 510, 515, 520, activators DIC 530, PIP 535, HBTU 540, and base 545, and washing solvents 555 may be transported by slug flow over the immobilized peptide or protein 575.

In other embodiments, amino acids or peptides may be immobilized on a resin 575, e.g., solid support. The resin 575 may be contained within a reaction vessel. In certain embodiments, a plurality of reagent reservoirs, e.g., amino acids 510, 515, 520, activators DIC 530, PIP 535, HBTU 540, and base 545, may be located upstream of and connected to the reactor (FIG. 5).

In some embodiments, a reagent reservoir contains amino acids or peptides, e.g., pre-activated amino acids or peptides and/or amino acids or peptides that are not fully activated. In certain embodiments, a reagent reservoir contains an amino acid activating agent, e.g., an alkaline liquid 545, a carbodiimide 530, and/or a uronium activating agent 540, capable of completing the activation of the amino acids 510, 515, 520.

In still other embodiments, a reagent reservoir contains a deprotection agent, e.g., piperidine 535 or trifluoroacetic acid. A reagent reservoir may contain a solvent 555, e.g., DMF that may be used in a reagent removal step. While single reservoirs have been illustrated in FIG. 5 for simplicity, it should be understood that in FIGS. 3 and 4, where single reservoirs are illustrated, multiple reservoirs, e.g., each containing different types of amino acids, different types of deprotection agents, etc., can be used in place of the single reservoir.

In certain embodiments, amino acids, peptides and proteins comprise protecting groups, for example, on the N-termini of the amino acid, peptide or protein. Protecting groups include chemical moieties that are attached to or are configured to be attached to reactive groups, e.g., C-terminus, N-terminus or side-chain, on the amino acids, peptides and proteins such that the protecting groups prevent the amino acid, peptide or protein from reacting. Protection may occur by attaching the protecting group to the amino acid, peptide or protein. Deprotection may occur when the protecting group is removed from the amino acid, peptide or protein, for example, by a chemical transformation by slug flow techniques described herein.

In other embodiments, a plurality of amino acids, peptides and proteins comprising protecting groups may be bound to a plurality of resins such that each amino acid, peptide or protein is immobilized on a resin. For example, the amino acids, peptides and proteins may be bound to the resin via their C-termini, thereby immobilizing the amino acid, peptide or protein.

In some embodiments, the process of adding amino acids or peptides to immobilized amino acids, peptides or proteins on the resin comprise exposing a deprotection agent to the immobilized amino acids, peptides or proteins to remove at least a portion of the protecting groups from at least a portion of the immobilized amino acids, peptides or proteins. The deprotection step can be configured, in certain embodiments, such that side-chain protecting groups are preserved, while N-termini protecting groups are removed.

In other embodiments, the protecting group used to protect the amino acids, peptides or proteins comprise Fmoc. In such embodiments, a deprotection agent comprising piperidine, e.g., a piperidine solution, may be exposed to the immobilized amino acids, peptides or proteins such that the Fmoc protecting groups are removed from at least a portion of the immobilized amino acids, peptides or proteins.

In still other embodiments, the protecting group used to protect the amino acids, peptides or proteins comprise Boc. In such embodiments, a deprotection agent comprising TFA may be exposed to the immobilized peptides such that the Boc protecting groups are removed from at least a portion of the immobilized amino acids, peptides or proteins. In certain instances, the protecting groups, e.g. Boc, may be bound to the N-termini of the peptides.

In certain embodiments, at least a portion of the reaction byproducts, e.g., protecting groups, that may have formed during the deprotection step can be removed by the slug flow techniques described herein.

In some embodiments, the deprotection agent and/or byproducts are removed by washing the amino acids, peptides or proteins, solid support, and/or surrounding areas with a washing solvent, e.g., a liquid such as an aqueous or non-aqueous solvent, a supercritical fluid, or the like. In other embodiments, removing the deprotection agent and reaction byproducts improves the performance of the slug flow technique, e.g., by preventing side reactions.

In other embodiments, the process of coupling protected amino acids or peptides to immobilized unprotected amino acids, peptides or proteins comprise exposing activated amino acids to the immobilized unprotected amino acids, peptides or proteins, such that at least a portion of the activated amino acids or peptides are coupled to the immobilized amino acids, peptides or proteins to lengthen the peptide or protein chain. For example, the peptides may be exposed to activated amino acids or peptides that react with the deprotected N-termini of the amino acids, peptides or proteins that are immobilized on the resin. In certain embodiments, amino acids or peptides can be activated for reaction by mixing the amino acid or peptide containing stream with an activation agent stream to form a slug, as disclosed herein.

In some embodiments, the method or system comprises removing at least a portion of the activated amino acids or peptides that do not bond to the immobilized amino acid, peptide or protein. At least a portion of the reaction byproducts that may form during the activated amino acid exposure step may be removed. In certain embodiments, the activated amino acids and byproducts are removed by washing with a slug of wash solvent.

It should be understood that the above-referenced steps are exemplary and an amino acid or peptide slug need not necessarily comprise all of the above-referenced steps. Generally, an amino acid or peptide slug includes any series of steps that results in the coupling of the protected amino acid or peptide to an unprotected amino acid, peptide or protein that is immobilized on the resin.

In certain embodiments, subsequent amino acid or peptide coupling by slug flow as described herein, can be used to lengthen amino acid, peptide or protein chains by adding amino acid or peptides individually until the desired peptide or protein has been synthesized.

In some embodiments, more than one amino acid or peptide may be added by slug flow to an amino acid, peptide or protein immobilized on a resin.

In some embodiments, amino acids or peptides can be coupled to amino acids, peptides or proteins immobilized on a resin by slug flow significantly faster than conventional semi-continuous or continuous manufacturing methods known in the art. The total amount of time taken to perform the slug flow can be influenced by the protecting group. For instance, when the desired protecting group comprises Fmoc, the total amount of time taken to perform the slug flow technique including the deprotection agent exposing step, the deprotection agent removal step, the activated amino acid exposing step, and the unreacted activated amino acid removal step is less than about 10 minutes, e.g., about 9 minutes, about 8 minutes, about 7 minutes, about 6 minutes, about 5 minutes, about 4 minutes, about 3 minutes, about 2 minutes, about 1 minute, about 50 seconds, about 40 seconds, about 30 seconds, about 20 seconds, about 10 seconds, about 7 seconds, about 5 seconds, or about 2 seconds.

In other embodiments, including embodiments in which the protecting groups comprise Boc, Fmoc, and/or other types of protecting groups, the total amount of time taken to perform the slug flow technique including the deprotection agent exposing step, the deprotection agent removal step, the activated amino acid exposing step, and the unreacted activated amino acid removal step is less than about 10 minutes, e.g., about 9 minutes, about 8 minutes, about 7 minutes, about 6 minutes, about 5 minutes, about 4 minutes, about 3 minutes, about 2 minutes, about 1 minute, about 50 seconds, about 40 seconds, about 30 seconds, about 20 seconds, about 10 seconds, about 7 seconds, about 5 seconds, or about 2 seconds.

In general, the total amount of time taken to perform the slug flow technique including the deprotection agent exposing step, the deprotection agent removal step, the activated amino acid exposing step, and the unreacted activated amino acid removal step can be decreased by inline analytics capable of making in-sequence optimizations, as described herein.

In certain embodiments, the first amino acid or peptide coupling step, and subsequent coupling steps proceed in high yields. For example, exposing activated protected amino acids or peptides by slug flow to the immobilized unprotected amino acid, peptide or protein results in coupling to the immobilized unprotected amino acid, peptide or protein in greater than about 95%, e.g., about 96%, about 97%, about 98%, about 99%, about 99.9%, about 99.99%, or 100% yield.

In some embodiments, a second amino acid or peptide coupling step, or subsequent third, fourth, fifth, and/or a subsequent coupling steps proceed in high yield using the methods and systems described herein. For example, exposing activated protected amino acids or peptides by slug flow to the immobilized unprotected amino acid, peptide or protein results in coupling to the immobilized unprotected amino acid, peptide or protein in greater than about 95%, e.g., about 96%, about 97%, about 98%, about 99%, about 99.9%, about 99.99%, or 100% yield.

In other embodiments, the methods and systems allow the coupling of greater than one amino acid or peptide to the immobilized amino acid, peptide or protein by the slug flow technique disclosed herein.

In other embodiments, one or more amino acids or peptides can be coupled to the unprotected amino acid or peptide that is immobilized on the resin with little or no double coupling, e.g., the addition of multiple copies of a amino acid or peptide during a single coupling step. For example, multiple copies of the amino acid or peptide are coupled to the immobilized amino acid, peptide or protein in less than about 1%, e.g., about 0.1%, about 0.01%, about 0.001%, about 0.0001%, or about 0.00001%, during the first, second, third, fourth, fifth, or subsequent steps of the methods or systems disclosed herein.

Certain steps in the amino acid or peptide slug flow technique may require mixing of reagents. In conventional systems, reagents are mixed for long periods of time prior to exposure to the immobilized amino acid, peptide or protein, which may result in undesirable side reactions and/or reagent degradation prior to exposure to the amino acids, peptides or proteins.

In some instances, the side reactions and/or degradation adversely affects the yield, kinetics and efficiency of the amino acid or peptide coupling. One technique for achieving rapid peptide or protein synthesis involves using slug flow to deliver and couple amino acids or peptides to the immobilized amino acids, peptides or proteins, as shown in FIGS. 3 and 6.

In some embodiments, a method or system for rapidly delivering and coupling amino acids or peptides to immobilized peptides or proteins comprises using slug flow to deliver a slug to a resin wherein the slug comprises: a) a protected amino acid or peptide; b) an activating agent, e.g., an alkaline liquid, a carbodiimide, and/or a uronium activating agent; and c) a solvent. For example, a reagent reservoir may comprise amino acids, e.g., 510, 515, 520, or peptides.

In certain embodiments, a reagent reservoir may comprise an amino acid or peptide activating agent, e.g., DIC 530, PIP 535, HBTU 540. The slug may comprise activated amino acids or peptides due to the activation of the amino acids or peptides by the activating agent. After the amino acids or peptides have been activated, the immobilized amino acids, peptides or proteins may be exposed to the slug within a relatively short period of time. For example, in some embodiments, the plurality of amino acids, peptides or proteins that are immobilized on the resin may be exposed to the slug in less than about 30 seconds, e.g., about 25 seconds, about 20 seconds, about 15 seconds, about 10 seconds, about 5 seconds, about 3 seconds, about 2 seconds, about 1 second, about 0.1 seconds, or about 0.01 seconds.

Another technique for achieving fast synthesis times may involve heating the slug prior to arriving to the reactor comprising the resin. Supplying the reactor with heat may alter the kinetics of the reaction occurring in the reactor. For example, exposing immobilized amino acids, peptides or proteins, solid supports, or other synthesis components to heat may alter the reaction kinetics and/or diffusion kinetics of the manufacturing process. For example, exposing the activated protected amino acids or peptides to heat may increase the rate at which the amino acids or peptides are coupled to the immobilized amino acids, peptides or proteins.

Thus, in certain embodiments, a method or system for coupling amino acids or peptides to immobilized amino acids, peptides or proteins comprise heating a slug of activated protected amino acids or peptides such that the temperature of the activated protected amino acids or peptides is increased by at least about 1° C., e.g., about 2° C., about 5° C., about 10° C., about 25° C., about 50° C., about 75° C., about 100° C., about 125° C., about 150° C., about 175° C., about 200° C., about 225° C., about 250° C., and about 275° C., or about 295° C. prior to being exposed to the immobilized amino acids, peptides or proteins.

In some embodiments, a slug comprising other components, e.g., a washing solution, a deprotection agent, or any other component, comprise heating a slug of activated protected amino acids or peptides such that the temperature of the activated protected amino acids or peptides is increased by at least about 1° C., e.g., about 2° C., about 5° C., about 10° C., about 25° C., about 50° C., about 75° C., about 100° C., about 125° C., about 150° C., about 175° C., about 200° C., about 225° C., about 250° C., and about 275° C., or about 295° C. prior to being exposed to the immobilized amino acids, peptides or proteins.

In other embodiments, the heating step, e.g., the heating of the slug comprising activated amino acids or peptides and/or the heating of other components transported to the immobilized amino acids, peptides or proteins, is performed in less than about 30 seconds, e.g., about 25 seconds, about 20 seconds, about 15 seconds, about 10 seconds, about 5 seconds, about 3 seconds, about 2 seconds, about 1 second, about 0.1 seconds, or about 0.01 seconds of exposing the slug contents, e.g., the heated activated protected amino acid or peptide to the immobilized amino acids, peptides or proteins. In such embodiments, as illustrated in the exemplary embodiments of FIGS. 4 and 5, heating may be achieved by heating a location upstream of the immobilized amino acids, peptides or proteins.

In certain embodiments, the heating of the slug comprising activated protected amino acids or peptides begins at least about 0.1 seconds, e.g., about 1 second, about 5 seconds, or about 10 seconds prior to exposing the slug comprising activated protected amino acids or peptides to the immobilized amino acids, peptides or proteins.

In still other embodiments, the slug comprising activated protected amino acids or peptides are heated by at least about 1° C., e.g., about 2° C., about 5° C., about 10° C., about 25° C., about 50° C., about 75° C., about 100° C., about 125° C., about 150° C., about 175° C., about 200° C., about 225° C., about 250° C., and about 275° C., or about 295° C. at least about 0.1 seconds, e.g., about 1 second, about 5 seconds, or about 10 seconds prior to exposing the slug comprising activated protected amino acids or peptides to the immobilized amino acids, peptides or proteins.

Referring back to FIGS. 3-6, for example, systems 300, 400, 500, 600 may comprise heating zones, which the contents of a slug may be heated.

In some embodiments, the heating zones may comprise a heater. In general, any suitable method of heating may be used to increase the temperature of a slug. For example, heating zones may comprise a liquid bath, e.g., a water bath, a resistive heater, a gas convection-based heating element, or other suitable heaters known in the art.

In other embodiments, the heating mechanism may be within a short distance of the immobilized amino acids, peptides or proteins, for example, within about 5 meters, e.g., about 1 meter, about 50 cm, or about 10 cm.

In other embodiments, including those illustrated in FIGS. 3-6, the heating of the slug comprising protected amino acids or peptides and activating agents, e.g., an alkaline liquid, a carbodiimide, and/or a uronium activating agent, can be performed before and during contact with the immobilized amino acids, peptides or proteins.

In certain embodiments, heating the slug prior to exposure to the immobilized amino acids, peptides or proteins, as opposed to heating the slug before transport to the immobilized amino acids, peptides or proteins, minimizes the thermal degradation of one or more reagents, e.g., the amino acids or peptides that are to be added to the peptides or proteins and/or the deprotection agent in the slug.

Approaches for reducing pressure across the immobilized amino acids, peptides or proteins may be used to improve the efficiency of the peptide or protein synthesis as described herein. In some embodiments, the flow rate of reagents across the immobilized amino acids, peptides or protein reduces the time of the synthesis. For example, the time required for slug flow delivery of an amino acid or peptide, activating reagents, and a solvent may decrease with increasing flow rate. Pressure drop may occur due to expansion of the resin during synthesis.

In other embodiments, the pressure drop across the resin during slug delivery of an amino acid or peptide does not exceed about 700 psi for more than about 5% during which the slug flow technique is performed.

The time required for peptide or protein synthesis is influenced by the choice of protecting group. For example, the use of Fmoc protecting groups is generally understood to require longer synthesis times. However, the methods and systems described herein can be used to perform rapid amino acid or peptide addition, even when Fmoc protecting group chemistry is employed. In general, any protecting group known to those of ordinary skill in the art can be used. Non-limiting examples of protecting groups, e.g., n-terminal protecting groups) include fluorenylmethyloxycarbonyl, tert-butyloxycarbonyl, allyloxycarbonyl (alloc), carboxybenzyl, photolabile protecting groups, or a combination thereof.

In some embodiments, immobilized amino acids, peptides or proteins comprise fluorenylmethyloxycarbonyl protecting groups. In other embodiments, immobilized amino acids, peptides or proteins comprise tert-butyloxycarbonyl protecting groups.

As previously described above, an amino acid activating agent may be used to activate or complete the activation of amino acids or peptides prior to exposing the amino acids or peptides to immobilized amino acids, peptides or proteins (FIGS. 4 and 5). Any suitable amino acid activating agent, e.g., DIC 530, PIP 535, HBTU 540, may be used.

In some embodiments, the amino acid activating agent comprises an alkaline liquid. In other embodiments, the amino acid activating agent comprises a carbodiimide, e.g., N,N′-dicyclohexylcarbodiimide (DCC), 1-ethyl-3-(3-dimethylaminopropyl)carbodiimide (EDC), or the like. In certain embodiments, the amino acid activating agent comprises a uronium activating agent, e.g., O-(Benzotriazol-1-yl)-N,N,N′,N′-tetramethyluronium hexafluorophosphate (HBTU), 2-(7-Aza-1H-benzotriazole-1-yl)-1,1,3,3-tetramethyluronium hexafluorophosphate (HATU), 1-[(1-(Cyano-2-ethoxy-2-oxoethylideneaminooxy) dimethylaminomorpholino)] uronium hexafluorophosphate (COMU), or a combination thereof.

Also previously described, peptides or proteins are immobilized on a resin, e.g., solid support. In general, any solid support may be used with any of the slugs described herein. Non-limiting examples of solid support materials include polystyrene, e.g., microporous polystyrene resin, mesoporous polystyrene resin, macroporous polystyrene resin, glass, polysaccharides, e.g., cellulose, agarose, polyacrylamide resins, polyethylene glycol, or copolymer resins, e.g., comprising polyethylene glycol, polystyrene, and the like. The resin can be in any suitable form. For example, the resin can be in the form of beads, particles, fibers, or other suitable forms known to one skilled in the art.

In some embodiments, the resin, e.g., solid support, may be porous. For example, in certain embodiments, macroporous materials, e.g., macroporous polystyrene resins, mesoporous materials, and/or microporous materials, e.g., microporous polystyrene resin, may be employed as a resin. The terms “macroporous,” “mesoporous,” and “microporous,” when used in relation to resins for peptide or protein synthesis, are known to those of ordinary skill in the art and are used herein in consistent fashion with their description in the International Union of Pure and Applied Chemistry (IUPAC) Compendium of Chemical Terminology, Version 2.3.2, Aug. 19, 2012 (informally known as the “Gold Book”). Generally, microporous materials include those having pores with cross-sectional diameters of less than about 2 nanometers. Mesoporous materials include those having pores with cross-sectional diameters of from about 2 nanometers to about 50 nanometers. Macroporous materials include those having pores with cross-sectional diameters of greater than about 50 nanometers and as large as 1 micrometer.

### Peptide Synthesis

Fully protected resin-bound peptides and proteins were synthesized via standard Fmoc solid-phase peptide chemistry and using an automated peptide synthesizer. All N-Fmoc amino acids were employed. Fmoc removal was achieved by treatment, e.g., 20% piperidine in DMF, followed by multiple (2 to 5) DMF washes. For all Fmoc amino acid couplings, the resin was treated with about 6 eq. of Fmoc amino acid, about 6 eq. of HBTU and about 9 eq. of DIPEA in NMP or DMF. For difficult couplings, a second treatment with about 6 eq. of Fmoc amino acid, about 6 eq. of HBTU and about 9 eq. of DIPEA in NMP was employed.

Once the peptide was synthesized, following Fmoc removal, the resin was treated with an appropriate cleavage solution, to afford cleavage from the solid support and/or deprotection. The purity of the crude peptide was then analyzed by reversed-phase LC-MS. In certain embodiments, the peptide was subsequently purified by, e.g., size-exclusion chromatography (SEC), strong cation exchange (SCX) chromatography, reversed-phase (RP) chromatography, other standard chromatography methods known in the art, or a combination thereof, and lyophilized to obtain an isolated product.

### EXAMPLES

### Example 1. Implementation of Slug Flow Based Solid Phase Peptide Synthesis to Produce Peptide Sequences

A solid phase synthesis system was modified to utilize a custom fluidic module where slugs were generated and delivered to the resin bed (FIGS. 4, 5 and 7). The fluidic manifold and slug production was evaluated to determine the residence time and mixing characteristics relative to flowrate. The residence time was calculated using step input of an amino acid tracer allowing the monitoring of the outlets' concentration within the reaction solvent. The utilization of amino acids and reaction solvent eliminated potential experimental errors relative to tracer and solvent effects. The UV detector was installed on the outlet of the amino acid tube where the quinione tracer was added. Slug formation was observed by installing a 10 uL slug between the injected dye at the beginning and ending through the selector valve. A 1000 uL injection volume of solution was used for the process. A UV detector was also placed on the waste line leaving the port position valve (585, FIG. 5).

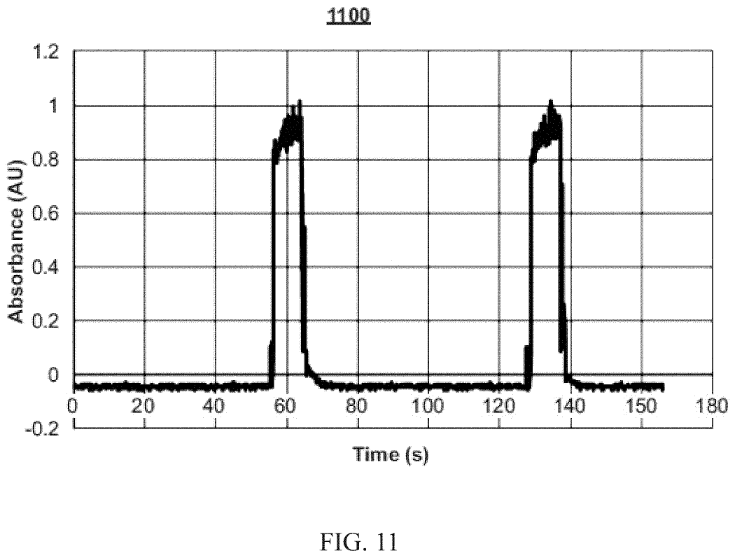

The reactor flow patterns in the reaction vessel was modified to allow for the delivery of slugs which was then evaluated to determine the synthesis reagent residence time and mixing characteristics relative to the flowrate, e.g., FIG. 6. The residence time was calculated using step input of an amino acid tracer, allowing the monitoring of the unreacted amino acid concentration in the reaction solvent at the reaction vessel outlet. The utilization of free amino acids and reaction solvent eliminated potential experimental errors relative to tracer and solvent effects. For evaluating mixing efficiencies, the Dushman reaction was used to determine mixing time. As shown in FIG. 11, slug formation 1100 has been established at an absorbance at 400 nm corresponding to the quinone dye through the amino acid valve, following a wash with multiple 10 uL slugs. In each case, it was established that mixing was occurring at the front of the slug.

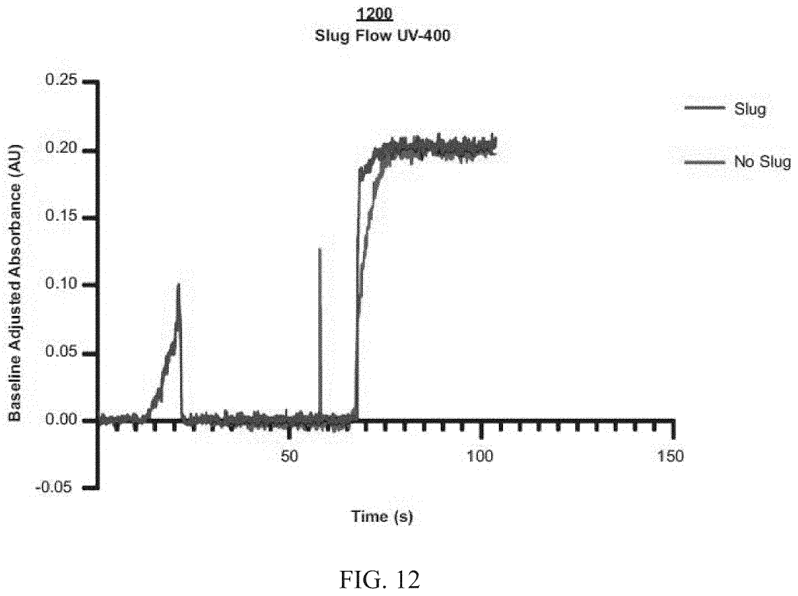

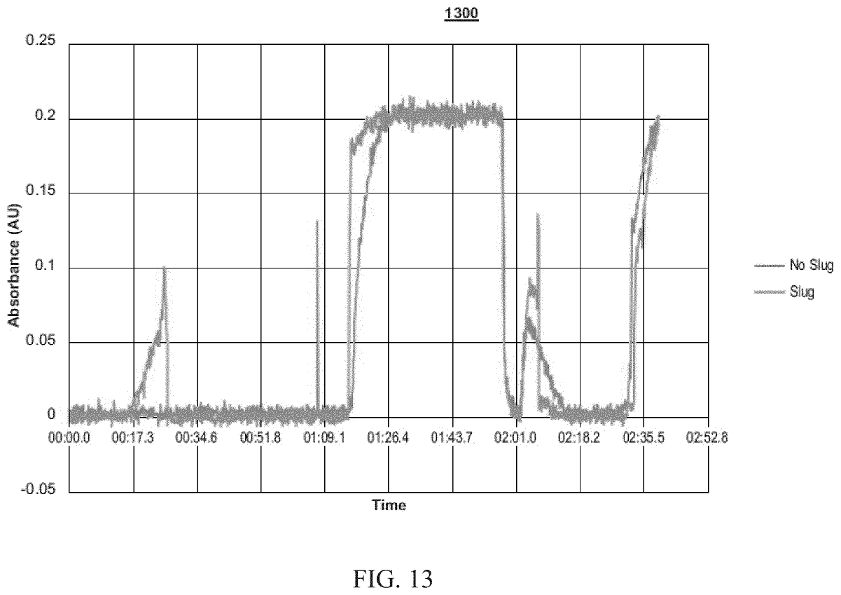

The slug flow reaction conditions were also evaluated with respect to residence and mixing time, e.g., FIG. 6. A narrow residence time and short mixing time was found to be desired to ensure rapid washout and fast reaction rates. These characteristics allowed for the selection and evaluation of the slug flow fluidic module mixing time, wash out volumes and residence times resulting in proper synthesis condition selection. In FIG. 12, the baseline was adjusted to an absorbance at 400 nm based on the quinone dye through amino acid valve and showed no slug formation versus slug formation in system 1200. These studies have been conducted more than one time to insure accuracy and optimization. FIG. 13 also shows the difference between no slug formation and slug formation in system 1300 when the baseline was adjusted to an absorbance at 400 nm based on the quinone dye through amino acid valve.

### Example 2. Synthesis of Peptides Based on Slug Flow

Simple peptides were synthesized using slug flow technology as described in Example 1. Simple peptides containing naturally occurring amino acids at varying base positions were synthesized to test the ability of coupling utilizing slug flow techniques. The solid phase synthesis system utilized inline analytics to monitor the individual amino acid coupling efficiency relative to length under various processing conditions. Utilizing the inline yield data, a 4×4 design of experiments (DOE) varying the temperature, flow rate, coupling volume, and coupling reagent was used to study the statistical relevance of system conditions on amino acid coupling efficiency relative to the length. Various peptides were coupled using solid phase synthesis based on slug flow technology, cleaved from the peptidyl resin using slugs, purified using slugs, and analyzed on HPLC-MS to compare purity and yield of the peptides produced. These studies demonstrated the ability of the slug flow system to efficiently couple amino acids using slug flow under optimal coupling conditions producing peptides in an efficient manner.

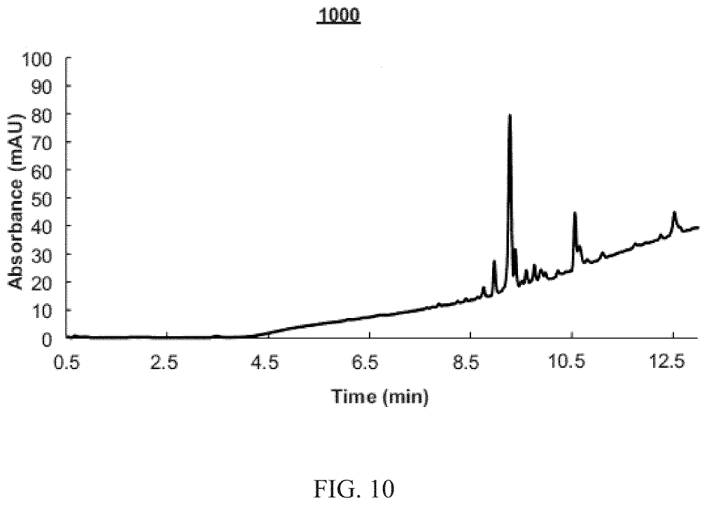

Based on inline analytical monitoring, the reaction yield relative to peptide length was observed in near real-time allowing statistical analysis to be conducted to understand the effect of the coupling condition variables on the growing peptide chain when slug flow was used. The inline and global measurements of yields allowed the determination of optimal synthesis conditions for conducting peptide synthesis utilizing the developed slug flow fluidic modules. The data collected from both inline PAT and LC-MS allowed real-time optimization models for subsequent synthesis. Moreover, the utilization of the LC-MS chromatograms allowed for the assessment of target peptide production and the determination of purity of the final peptide product. Subsequent statistical analyses were then utilized to evaluate the effects of the slug flow technique on reaction yield and purity. FIG. 10 shows chromatogram 1000 obtained from the inline UV data from the reactor outlet for GIFGIF at UV 220 nm. Based on FIG. 10, the implementation of slug flow based solid phase peptide synthesis to efficiently produce peptides have been accomplished with a higher degree of control and unwanted mixing and diffusion.

### Example 3. Slug Flow Synthesis of Acyl Carrier Protein (ACP(65-74))

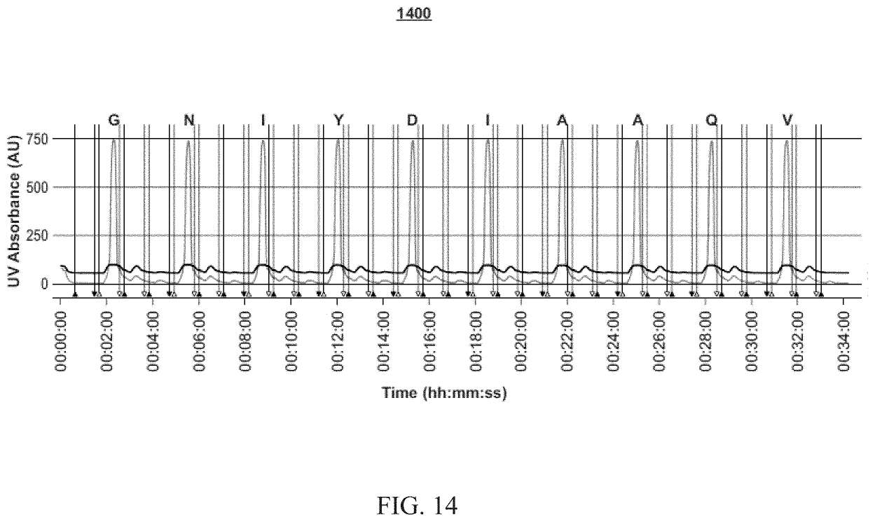

To evaluate the efficacy of the methods and systems based on slug flow synthesis, acyl carrier protein (ACP(65-74)) was synthesized using slug flow according to Examples 1 and 2. Acyl carrier protein (ACP(65-74)) was synthesized using the optimized slug flow conditions as established in Examples 1 and 2. Following synthesis by slug flow coupling, cleavage of ACP(65-74) from the resin was accomplished using a trifluoroacetic acid slug. Analytical purification method 1400 was performed and the samples was analyzed using HPLC-MS to determine the retention time and mass to verify that the target peptide was produced FIG. 14. FIG. 9 shows chromatogram 900 from the inline UV data from the reactor outlet for acyl carrier protein (ACP(65-74)) at UV 220 nm. ACP sequence amino acids 65-74; 13-mer) amino-VQAAIDYINGHIV-carboxy; m/z [M+H]⁺ for C47H75N13O15 1062.5; Final purity (210 nm): >98%. This result was shown to be efficient based on inline PAT during the slug flow peptide synthesis and evaluation with HPLC-MS.

### Example 4. Slug Flow Synthesis of Pexiganan

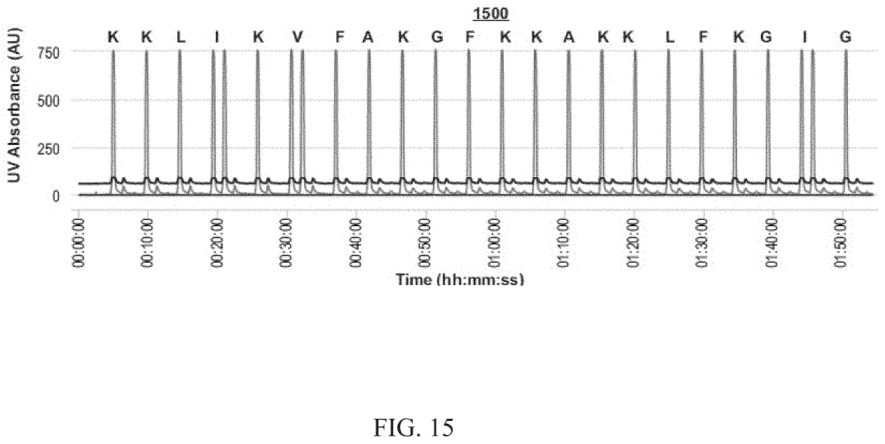

To evaluate the efficacy of the methods and systems based on slug flow synthesis, pexiganan was synthesized using slug flow according to Examples 1 and 2. Pexiganan was synthesized using the optimized slug flow conditions as established in Examples 1 and 2. Following synthesis by slug flow coupling, cleavage of pexiganan from the resin was accomplished. Analytical purification method 1500 was performed and the samples was analyzed using HPLC-MS to determine the retention time and mass to verify that the target peptide was produced FIG. 15. Pexiganan sequence GIGKFLKKAKKFGKAFVKILKK, C-Terminus=Amide, N-Terminus=NH2; m/z [M+2H]⁺ C122H210N32O22 1239.3; Final purity (210 nm): >91%. This result was shown to be efficient based on inline PAT during the slug flow peptide synthesis and evaluation with HPLC-MS.

### Example 5. Slug Flow Synthesis of Glucagon-Like-Peptide-1 (GLP-1)

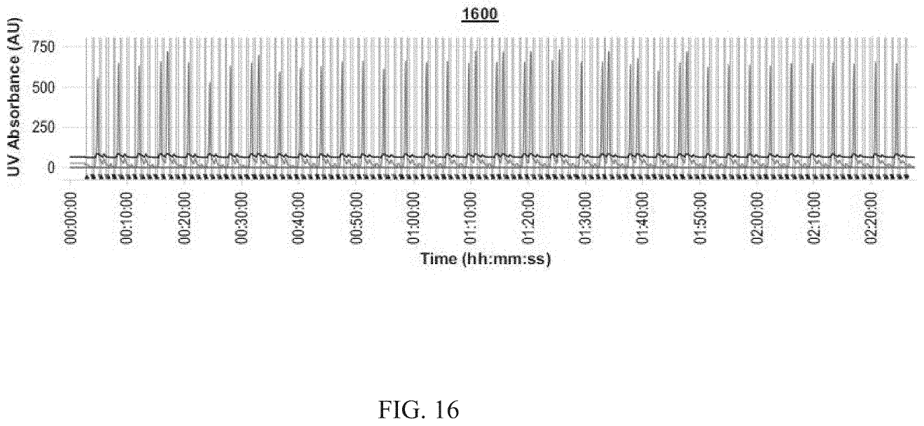

To evaluate the efficacy of the methods and systems based on slug flow synthesis, glucagon-like-peptide-1 (GLP-1) was synthesized using slug flow according to Examples 1 and 2. GLP-1 was synthesized using the optimized slug flow conditions as established in Examples 1 and 2. Following synthesis by slug flow coupling, cleavage of GLP-1 from the resin was accomplished. Analytical purification method 1600 was performed and the samples was analyzed using HPLC-MS to determine the retention time and mass to verify that the target peptide was produced FIG. 16. m/z [M+3H]+C187H275N47O57 1365.2. This result was shown to be efficient based on inline PAT during the slug flow peptide synthesis and evaluation with HPLC-MS.

### Example 6. Slug Flow Synthesis of Beta Amyloid

To evaluate the efficacy of the methods and systems based on slug flow synthesis, beta amyloid was synthesized using slug flow according to Examples 1 and 2. Beta amyloid was synthesized using the optimized slug flow conditions as established in Examples 1 and 2. Following synthesis by slug flow coupling, cleavage of beta amyloid from the resin was accomplished. An analytical purification method was performed and the samples was analyzed using HPLC-MS to determine the retention time and mass to verify that the target peptide was produced. Beta amyloid sequence amino-DAEFRHDSGYEVHHQKLVFFAEDVGSNKGAIIGLMVGGVVIA-carboxy; m/z [M+3H]⁺ C203H312N56O59S1 1505.1. This result was shown to be efficient based on inline PAT during the slug flow peptide synthesis and evaluation with HPLC-MS.

### Example 7. Optimization of the Slug Flow System for Decreased Dispersion and Mixing Demonstrating Real-Time Feedback

Computation: Inline measurements of conductivity and UV-VIS absorption at all wavelengths at the flow path outlet was maximized by evaluating the extent of coupling by calculating the difference between measured and expected “no reaction” amino acid concentration; and calculating the extent of deprotection by measuring the dibenzofulvene-piperidine adduct concentration.

The flow rate, flow duration, temperature, and sequence/repetition of steps constitute the real-time tunable handles of the slug flow system. Based on the above measurements for a step, e.g., the extent of deprotection, a decision was made for the next step, e.g., repeating the deprotect due to incomplete initial reaction, enabling improvement in the amino acid to amino acid processing. Real-time analysis was further used to reduce risk by reducing recovery time and waste generation by prematurely terminating and restarting synthesis in response to detected failed reactions.

The use of the real-time optimization approach vs. the fixed standard operating procedure demonstrated reduced synthesis time by 50% and reduced impurity by 50% for members of the peptides shown in Examples 1 and 2.

### Example 8. The Building of End-to-End Machine Learning Models to Predict Peptide Synthesis Properties for Process Optimization

The development of machine learning (ML) models has been accomplished to optimize the coupling process to increase purity, to decrease reaction time, and to reduce waste using integrated process analytical technologies. End-to-end machine learning has enabled advancements in image recognition and language translation. These methods have also been applied to offline collected data and pre-synthesis pipelines, and to online amino acid to amino acid predictions and optimization.

Computation: The data acquired from the previous Examples have been stored for offline analytics and for the training of ML models which consisted of “inputs”, e.g., the parameters of the peptide synthesis protocol plus the peptide sequence and derived features, and “outputs”, e.g., quantities to maximize yield and purity and quantities to minimize aggregation propensity, side product formation, and purification difficulty.

The predictors for the “outputs”, e.g., training on the full set of available synthesis data and validation of held out portions, have been built. Initially, simple models such as logistic regression and random forests have been used to predict categorical outputs, e.g., un/acceptable yield from the basic protocol parameters and the presence of amino acids in the sequence. Subsequently, more complex models such as deep neural nets were used to predict continuous outputs such as a combined “synthesizability” score from more complete sets of protocol parameters and raw representations of peptide composition. Expensive model training using GPU and cloud computing resources running state-of-the-art machine learning tools such as XGBoost and PyTorch was accomplished. Fast model inference was performed locally prior to each peptide synthesis. The models were retrained and revalidated as new peptides were synthesized and the data collected which allowed the prediction of successful first time peptide synthesis, e.g., yield and purity>above cutoffs, using a protocol selected from a set of default protocols with AUC >0.9 for a library of peptides.

### Example 9. The Use of Machine Learning Models to Optimize Peptide Synthesis Protocols

Computation: The general framework for using predictive models to improve peptide synthesis was accomplished by predicting synthesizability under a variety of possible simulated conditions prior to synthesis, selecting the conditions with the highest probability of success/most favorable results, and then performing the protocol. After performing the synthesis, the measured result was recorded, the prediction quality evaluated, and the results were added to the running dataset to inform future predictions. Small numbers of model evaluations were run locally. Large scale predictions were run on GPUs/cloud resources. The synthesizability metrics corresponded to the end-to-end model outputs outlined in Example 8. Initially a probability of successful target yield was achieved, followed by a score corresponding to a multi-objective optimization of desired outputs.

To assess consistency, rank concordance between predicted and actual protocol results was calculated. To assess performance, predicted optimal protocols was carried out in parallel with the unoptimized standard protocol, and better results in the predicted protocol indicated success. These data sets were repeated for a library of peptides.

### Example 10. Bioequivalence for Synthesized Peptides in Antimicrobial Assays

Validated antimicrobial peptides, e.g., E16LKL, T7 Novispirin, Myxinidin (Y8), WMD4R, C18AA, Pexiganin, Adepantin-1, Pint, SHc-CATH, and LL-37 Pentamide, can be produced using the SPSF coupling technology described in Examples 1 and 2. A colony-forming assay was used with E. coli and Streptococcus pyogenes to determine the minimum inhibitory concentration, e.g., the lowest concentration that shows 80% growth inhibition, for each synthesized and commercially purchased peptide sequence. Analysis consisted of colony counting using an open source software. When plated with commercial peptides assessing minimum inhibitory concentrations, a similarity within 10% in the colony counts for each of the ten synthesized peptides and their corresponding commercial analogs were considered to be successful.

A modified colony-forming assay as set forth by Oppenheim et al (2003) was used to analyze the biological activity of peptides accessed by using slug flow. Test microorganisms (Escherichia coli and Streptococcus pyogenes) were mixed with the antibiotic peptides obtained from Example 4 and was assessed at concentrations from about 0-25 micrograms per milliliter (μg/mL) in a culture media comprising of 0.1 mL 10 millimolar (mM) potassium phosphate buffer (pH 7.4) and 1% trypticase soy broth (TSB). The culture media/peptide/microorganism solution (“test solution”) was incubated for 3 hours at 37° C. in an environmental shaking incubator (250 rpm). The test solution was then serially diluted with 10 mM phosphate buffer (pH 7.4) containing 1% TSB and plated in triplicate on lysogeny broth (LB) plates. The LB plates were then incubated for approximately 20 hours at 37° C. and colonies were counted, by methods known in the art.

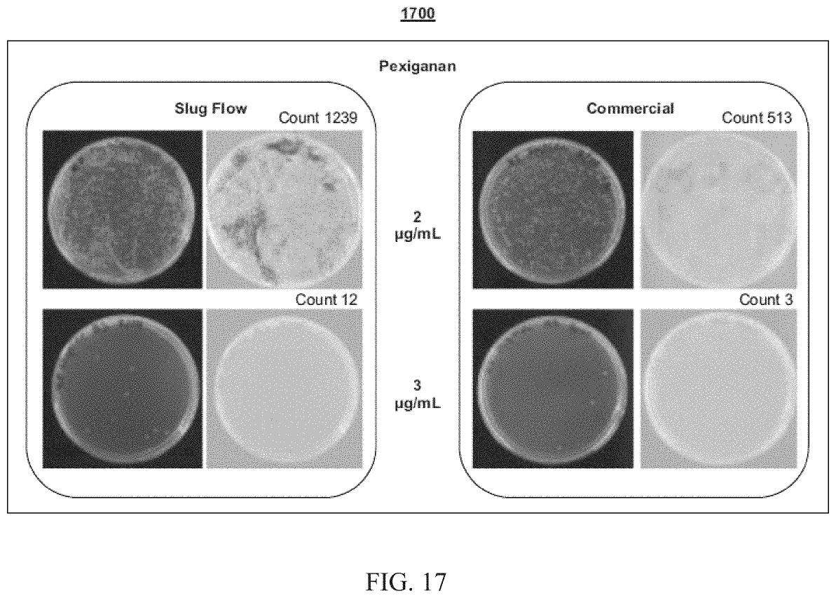

Modified colony-forming assay 1700 allowed for the assessment of antimicrobial properties of the synthesized peptides when compared to positive controls. The lowest peptide concentration resulting in 80%+/−10% growth inhibition relative to the positive control was considered for these purposes and to be the minimum inhibitory concentration as shown in FIG. 17. In all cases, the synthesized peptides demonstrated biological performance consistent with leading commercial vendor activity. FIG. 17.

### Example 11. Bioequivalence of Synthesized GLP-1 Peptide in a Live Cell Assay

Glucagon-like-peptide-1 (GLP-1) is a peptide hormone released in the small intestines that enters circulation and subsequently binds to the GLP-1 receptor of pancreatic beta cells, triggering an increase in cAMP and insulin levels. Given the ability of GLP-1 to induce insulin secretion, the peptide has been an active area of research. Thus, GLP-1 represents an attractive starting point model system for live cell assay as the ligand-receptor biology is well-established. A commercial kit served as the representative model of the binding of GLP-1 to receptors of pancreatic cells. GLP-1 was produced using the SPFS coupling technology as shown in Example 5. Synthesized GLP-1-receptor interaction was compared to an independent, vendor-prepared GLP-1, using a commercially available cellular assay. The peptide receptor interaction was assessed after 3 minutes of incubation using the commercial kit's chemiluminescence and fluorescence outputs. Comparison of outputs from peptide produced using SPSF coupling versus vendor peptide enabled evaluation of bioequivalence. A preliminary assay kit was used to determine the appropriate concentration of ligand to add to the wells. A similarity within 20% in both the fluorescence and chemiluminescence signals for the synthesized peptide compared to the five commercial peptides was considered to be successful.

### Example 12. High-Throughput, Low-Cost Library Synthesis of GLP-1 Mutants to Probe Effects on Receptor Binding

Manandhar and Ahn recently reviewed GLP-1 mutants that have been studied and concluded that there exists a lack of understanding of the binding and peptide-receptor interaction. In the case of GLP-1 amino acids, variation at Ala²⁴, Ala²⁵, Ala³⁰, and Trp³¹ have not been extensively studied despite their presence in the C-terminal binding domain. As negative controls, the modification of His⁷ with Ala and Asp¹⁵ for Arg have shown to significantly damage receptor binding. SPSF was used to efficiently generate a mutation library. These studies have demonstrated the ability of SPSF to quickly generate a library based design, through efficient synthesis, purification, and cellular assay analysis based on methods known in the art.

### Example 13. Slug Flow Synthesis of a 102-Mer with Endcapping

A 102-mer protein was synthesized using the optimized slug flow conditions as established in Examples 1 and 2, e.g., deliver piperidine to activation coil and then reactor; load sample loop with piperidine for deprotection; deliver piperidine to activation coil and then reactor; load sample loop with piperidine for deprotection; deliver acetic acid to activation coil and then reactor; load sample loop with 0.4 M acetic acid; deliver amino acid to activation coil and then reactor; load sample loop with 0.4 M Fmoc-amino acid. Following synthesis by slug flow coupling with endcapping, cleavage of the 102-mer protein from the resin (0.416 mmol/g rink amide resin, 100° C. reactor temperature) was accomplished using a trifluoroacetic acid slug under oscillation. Endcapping was performed by an additional coupling step prior to each set of deprotection steps where instead of filling the amino acid loop with amino acid, e.g., 0.4 M Fmoc-amino acid, the loop was filled with 0.4 M acetic acid in DMF.

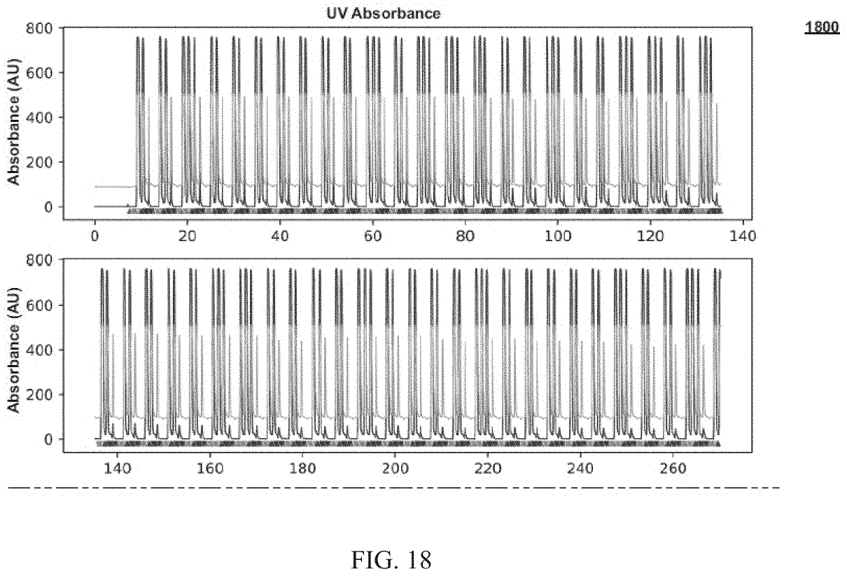

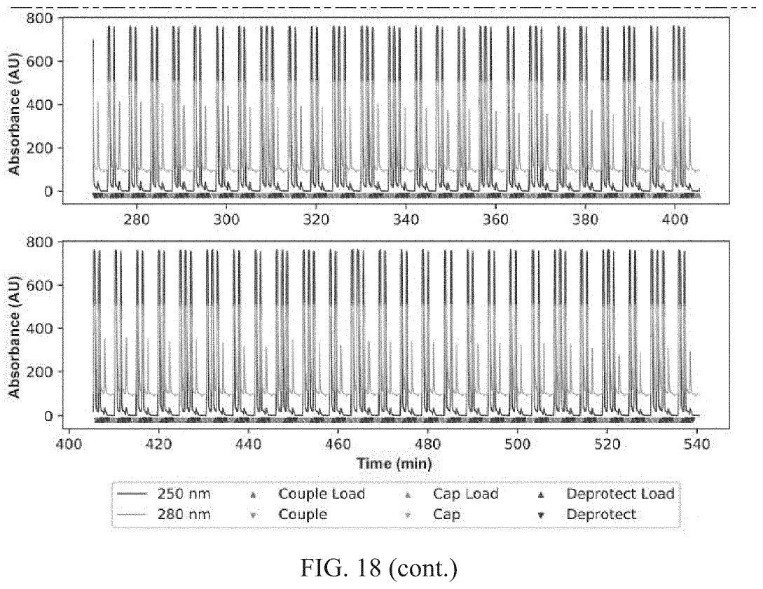

Analytical purification method 1800 was performed and the sample was analyzed using HPLC-MS to determine the retention time and mass to verify that the target protein was produced FIG. 18. FIG. 18 shows chromatogram 1800 from the inline UV data from the reactor outlet for the 102-mer protein at UV 210 nm. Protein synthesis was also confirmed by Total Ion Chromatogram (TIC). Sequence: MYGKLNDLLEDLQEVLKNLHKNWHGGKDNLHDVDNHLQNVIEDIHDFMQGGGSGGKLQEMMKEFQ QVLDELNNHLQGGKHTVHHIEQNIKEIFHHLEELVHR; C-Term=NH2; N-Term=NH; m/z Actual (Expected, Charge State): 1988.7 (1988.82, M+6), 1704.5 (1704.85, M+7), 1491.7 (1491.87, M+8), 1326.1 (1326.21, M+9), 1193.6 (1193.69, M+10), 1085.2 (1085.27, M+11), 995.0 (994.91, M+12), 918.5 (918.46, M+13), 852.9 (852.92, M+14), 796.0 (796.13, M+15), 746.4 (746.43, M+16), 702.4 (702.58, M+17), 663.6 (663.60, M+18). This result was shown to be efficient based on inline PAT during the slug flow peptide synthesis and evaluation with HPLC-MS.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1-208. (canceled)

209. A method for producing a peptide using semi-continuous or continuous manufacturing comprising inline analytics and solid phase peptide synthesis (SPPS),
wherein a plurality of reagents is delivered to a resin by semi-continuous or continuous manufacturing,
wherein the reagents comprise: a) a protected amino acid or peptide; b) an activating agent; and c) a solvent,
thereby coupling the protected amino acid or peptide to an unprotected amino acid or peptide that is immobilized on the resin.

210. The method of claim 209, wherein the reagents further comprise a base.

211. The method of claim 209, wherein the reagents further comprise a chaotropic agent.

212. The method of claim 209, wherein the reagents further comprise a deprotection agent, thereby deprotecting the peptide that is immobilized on the resin, and optionally elongating the peptide by coupling one or more amino acids or peptides sequentially to the peptide that is immobilized on the resin.

213. The method of claim 209, wherein the activating agent comprises HATU, HBTU, TBTU, DEPBT, PyAOP, PyBOP, DIC, DCC, COMU, HOBt, OBt ester, ODhbt ester, or a combination thereof.

214. The method of claim 209, wherein the solvent comprises dimethyl formamide (DMF), N-methylpyrrolidone (NMP), dimethyl sulfoxide (DMSO) and dichloromethane (DCM), acetonitrile, water, methanol, ethanol and isopropanol (2-propanol), n-propanol, n-butanol, isobutanol, sec-butanol, tert-butanol, amyl alcohol, heptanol, octanol, nonanol, decanol, hexanol, dioxane, tetrahydrofuran, diglyme, or a combination thereof.

215. The method of claim 211, wherein the chaotropic agent comprises n-butanol, ethanol, guanidinium chloride, lithium perchlorate, lithium acetate, magnesium chloride, phenol, 2-propanol, sodium dodecyl sulfate, thiourea, urea, trifluoroethanol, lithium bromide, DMSO, potassium thiocyanate, Triton X-100, or a combination thereof.

216. The method of 209, wherein the semi-continuous or continuous manufacturing comprises slug flow.

217. The method of claim 209, wherein the peptide is produced using semi-continuous manufacturing, wherein the semi-continuous manufacturing comprises slug flow.

218. The method of claim 209, wherein the peptide is produced using continuous manufacturing, wherein the continuous manufacturing comprises slug flow.

219. The method of claim 209, wherein the method further comprises subjecting to oscillation.

220. The method of claim 219, wherein the oscillation decreases washout time.

221. The method of claim 220, wherein the oscillation further decreases mixing time.

222. The method of claim 221, wherein the oscillation further decreases the coupling time of the protected amino acid or peptide to the unprotected amino acid or peptide that is immobilized on the resin.

223. The method of claim 209, wherein the method comprises reaction conditions comprising: a) concentrations; b) residence time; c) mixing time; d) temperature; e) flowrate; or a combination thereof.

224. The method of claim 223, wherein the residence time is calculated using step input of an amino acid tracer, allowing the monitoring of the unreacted protected amino acid or peptide concentration within the reaction solvent at the outlet of the resin.

225. The method of claim 209, wherein the inline analytics comprise: a) measuring reaction conditions; b) measuring reaction yields; c) measuring the effectiveness of the solid phase peptide synthesis (SPPS); d) measuring the temperature; or a combination thereof.

226. The method of claim 209, wherein a 60 amino acid peptide is formed in less than about 2 days.

227. The method of claim 209, wherein the peptide yield is a purity of at least about 70%, about 80%, about 90%, about 95%, or about 99% for a peptide from about 5 to about 100 amino acids in length.

228. A system for producing a peptide using semi-continuous or continuous manufacturing comprising inline analytics and solid phase peptide synthesis (SPPS),
wherein a plurality of reagents is delivered to a resin by semi-continuous or continuous manufacturing,
wherein the reagents comprise: a) a protected amino acid or peptide; b) an activating agent; and c) a solvent,
thereby coupling the protected amino acid or peptide to an unprotected amino acid or peptide that is immobilized on the resin.
